(19) United States
(12) Patent Application Publication
(10) Pub. No.: US 2005/0215889 A1
(43) Pub. Date: Sep. 29, 2005

(54) Methods for using pet measured metabolism to determine cognitive impairment

(75) Inventor: James C. Patterson II, Shreveport, LA (US)

Correspondence Address: Mark R. Wisner;c/o Wisner & Associates, Suite 400, 1177 West Loop South, Houston, TX, 77027, US

(73) Assignees: The Board of Supervisory of Louisiana State University; Agricultural and Mechanical College a Louisisana public consitutuional corporation

(21) Appl. No.: 10/811,633

(22) Filed: Mar. 29, 2004

(51) Int. Cl. A61B 5/05

(52) U.S. Cl. 600/436

(57) ABSTRACT

A non-invasive, early stage method to obtain quantitative measures of mild cognitive impairment useful in diagnosing and following degenerative brain disease or closed head injuries by utilizing the image data from individual patient positron emission tomographic scans to construct a cognitive decline index that serves as a diagnostic and screening tool to reveal the onset of mild cognitive impairment and nervous system dysfunction which are sequelae of degenerative brain diseases and closed head injury. The method involves using weighted values of brain region intensities derived from comparing scans of normal subjects to a scan of the patient to calculate a cognitive decline index that is useful as a diagnostic tool for mild cognitive impairment. The weights for the intensity values for each region are derived from the differences of intensity values from regions of the brain of the patient selected by comparing the patient to normal control subjects.

### BACKGROUND OF THE INVENTION

Diagnostic imaging and radiology began as a medical sub-specialty in the first decade of the 1900's after the publication in 1898 describing experiments on x-rays by Professor Wilhelm Roentgen. The development of radiology grew at a steady rate until World War II. Extensive use of x-ray imaging during the Second World War, and the advent of the digital computer and new imaging modalities like ultrasound, magnetic resonance imaging, single photon emission computed tomography and positron emission tomography have combined to create an explosion of diagnostic imaging techniques in the past 25 years.

In general, radiological imaging can address two issues: structure and function. It is possible either to view structures in the body and image anatomy or view chemical processes and image biochemistry. Structural imaging techniques can image anatomy and include ultrasound, X-rays, computerized axial tomography (CAT) and magnetic resonance imaging (MRI). Bone can be distinguished from soft tissue in X-ray imaging, and organs become delineated in CT and MRI imaging. All of the technologies described above have contributed to a foundation of extraordinary strength and breadth in diagnostic radiology. However, they all share the same basic limitation. The above described technologies reveal only anatomical structure. Pathology, whether injury, degeneration, lesion, tumor or anomaly, is revealed to the radiologist's trained eye as a deviation from normal structure.

Single photon emission computed tomography (SPECT) and positron emission tomography (PET)—differ from structural imaging modalities in that they follow actual chemical substituents and trace their routes through the body. These methods give functional images of blood flow and/or metabolism that are essential to diagnoses and to research on the brain, heart, liver, kidneys, bone and other organs of the human body. Since anatomical structures usually serve different functions and embody different biochemical processes, to some degree, biochemical imaging can provide anatomical information. However, the strength of these methods is to distinguish tissue according to metabolism not structure.

PET is an imaging technology that allows physicians and researchers to observe and analyze the chemical functioning of an organ or tissue, rather than anatomical structure as in MRI and CT. By examining cellular and metabolic activity, this imaging tool is vital to diagnosing and assessing the progression of diseases such as cancer, Parkinson's disease, Alzheimer's disease, heart disease, stroke and numerous other common afflictions. Furthermore, in research, PET allows for continuous and immediate monitoring of the effectiveness of medications and drugs under development.

PET consists of the systemic administration to the subject of a selected radiopharmaceutical labeled with one of several “physiological” radionuclides, 11C, 13N, 15O or 18F, followed by the measure, as a function of time, of the distribution of that nuclide in the structure of interest. The isotope 18F, generally in the form of 18F-fluoro-deoxyglucose (FDG), is particularly useful in neuroimaging because glucose metabolism is a clear indicator of changes in brain metabolism. The brain uses glucose as its only source of fuel unless in a state of starvation. The brain is very active metabolically, even during sleep. PET can be used to study the activity of the brain, because the amount of glucose metabolism in a given region will vary based on the activity of that region. The radioligand FDG is an analog of glucose and is taken up by brain cells just like glucose, but its metabolite is trapped in the cell. Thus, the more active the cell or region, the more radioactive glucose builds up, resulting in emission of more positrons from that region relative to other regions. Conversely, regions with decreased cellular activity have decreased metabolism, and decreased positron emission. The distribution is measured through the detection of the penetrating radiation emitted as a result of the annihilation of the positrons emitted from FDG.

These radionuclides are unstable because their nuclei contain an excess of protons with respect to a more stable configuration and decay to emit their excess positive charge in the form of positrons. Positrons are the anti-particles of electrons with the same rest energy, 511 million electron volts (mev) and charge of e+. When emitted, positrons travel a very short distance through matter and most probably bind with an electron forming, for a very brief time, a compound called positronium. Positronium decays very rapidly through annihilation into paired gamma rays each with energy of 511 mev and traveling in opposite directions. The energy partition between the gamma rays and their opposite direction of travel is necessary to conserve the energy and momentum of the original positronium system. The extremely short travel distance of the positron and its rapid initiation of the paired, equal energy gamma rays mean that the origin of the gamma rays can be assumed to be essentially the point of emission of the original positron. This fact and the short half-life of 18F of 110 minutes make this isotope a useful biological tracer permitting relatively large doses of activity with tolerable radiation exposure of the subject. The simultaneous emission of the paired, equal energy gamma rays traveling collinearly in opposite directions may be detected by paired photon detectors connected by a “coincidence” circuit that allows registration of an annihilation event only if the two photons detected on opposite sides of a subject impinge their separate detectors within a specified time period. This system provides an “electronic” form of collimation for the photons emitted from the annihilation event because it is sensitive to annihilation events occurring within a volume circumscribed by the straight line joining the two photon detectors and insensitive to events occurring outside this volume. The advent of coincidence detection of annihilation generated photons has led to images of greater quality and much finer resolution of the matter in which the annihilation event occurred.

There are a number of brain disorders that may be analyzed using functional PET functional imaging. These include degenerative brain. disorders such as Alzheimer's Disease (AD), Jacob-Kreutzfeldt disease and cerebral dysfunction caused by stroke, drug abuse and closed head injury. These diseases and conditions all show diminution of cognitive ability, loss of memory and may also show personality disorder. Measurement of cognitive decline or dysfunction is a powerful tool that can be used to identify, monitor and identify changes in these conditions. This cognitive decline or dysfunction is referred to throughout the instant patent as mild cognitive impairment (MCI).

One of the most feared medical problems for an individual to face is Alzheimer's disease (AD). Most patients know that there is little that can be done to slow the brain degeneration down, and nothing currently can be done to stop its course or prevent it. While available medications help, there is no cure, and a diagnosis of AD often means a long and troubled course. AD is the most common cause of dementia in late life, present in approximately 10% of those 65 years and older, and almost 50% of those 85 and older (Evans D A, Funkenstein H H, Albert M S, Scherr P A, Cook N R, Chown M J, Hebert L E, Hennekens C H, Taylor J O. (1989). Prevalence of Alzheimer's disease in a community population of older persons. Higher than previously reported. JAMA, 262(18):2551-6).

While these numbers are concerning enough, the prevalence in our aging population is increasing, and is projected to quadruple in the next half-century (Brookmeyer R, Gray S, Kawas C. (1998). Projections of Alzheimer's disease in the United States and the public health impact of delaying disease onset. Am J Public Health. 88(9): 1337-42).

Alzheimer's disease (AD) is one of the clinically most important amyloid disorders. AD currently ranks as the fourth “most expensive” disease in the United States, behind heart disease, cancer and diabetes. However, by 2010 costs associated with the care and treatment of seniors with AD in the US are expected to be greater than the costs associated with treating cancer and diabetes.

In 1990, 4 million people had AD, and this is expected to reach 14 million by 2050 (Katzman R, Kang D, Thomas R. (1998). Interaction of apolipoprotein E epsilon 4 with other genetic and non-genetic risk factors in late onset Alzheimer disease: problems facing the investigator. Neurochem Res. 1998 March;23(3):369-76.) Annual costs for patient care in 1998were $40,000 per patient (Petersen R C, Stevens J C, Ganguli M, Tangalos E G, Cummings J L, DeKosky S T. (2001). Practice parameter: early detection of dementia: mild cognitive impairment (an evidence-based review). Report of the Quality Standards Subcommittee of the American Academy of Neurology. Neurology. 56(9):1133-42.) Thus in 2050, a conservative estimate of annual costs is $560 billion dollars for patient care alone.

Alzheimer's disease (AD) is representative of a number of diseases result from chronic, pervasive processes that begin well before memory loss and concomitant cognitive impairment is noticed by the patient. In addition to AD, these diseases include Parkinson's disease, Huntington's disease, Pick's Dementia, Jakob Kreutzfeldt syndrome and Dementia with Lewy bodies. Mild cognitive impairment (MCI) resulting from head injury, patient intake of drugs or intake of alcohol round out a constellation of conditions that will benefit from diagnostic methods that will indicate the earliest possible detection.

Current methodologies for early detection and diagnosis of MCI and the degenerative diseases which induce MCI early in their development take several approaches, including genetic analysis, neuropsychological tests, and functional neuroimaging. Measurement of brain metabolism in vivo has been shown to be a very sensitive method to detect even early cognitive changes. In fact, several previous reports indicate that it is possible to detect brain functional changes across groups of patients before subjective symptoms or neuropsychological impairment occurs (Small GW, et al, (2000). PNAS 97(11):6037-6042, Reiman E M, et al, (2001). PNAS 98(6):3334-3339, De Leon M J, et al, (2001). PNAS 98(19):10966-10971). However, development of reliable methods of detecting MCI in individual patients at a clinical level is lacking. Detection of this degenerative process in premorbid states would enable the early treatment with medication to enhance and prolong quality of life, to provide an answer to the patients' questions regarding their potential for cognitive decline, to help them plan and prepare for the future, and hopefully one day to prevent the disease altogether.

There have been numerous efforts to utilize structural and functional imaging to diagnose, understand and monitor treatment of AD and related brain dysfunction or deterioration. A description of some of the more productive efforts will serve to illustrate the advancement of the teaching of the instant invention.

U.S. Pat. No. 5,262,945, DeCarli, et. al. Nov. 16, 1993 entitled, “Method for quantification of brain volume from magnetic resonance images” presents some of the earliest work on technology generally known as automated image segmentation. The DiCarli patent addresses the problem inherent in the technology that measuring volume of a structure in the brain had to do be done manually. This involved drawing, for example, two lines across a ventricle in a given brain image slice, in a cross formation, and then calculating volume based on those distances. It was possible and quite time consuming to outline the entire volume in that slice. Accuracy improved with more slices taken, however, the distinct boundary between ventricle and brain tissue had to be drawn by hand. Utilization of the properties of the digital images delivered by MRI enabled differentiation between tissues based on the difference of image intensities. Image quality and contrast led to the development of methods to select one pixel in the image (a seed-point) which was then used to automatically define a region based on contiguous pixels of a similar intensity. This in turn led to the ability to implement software to automatically place seed-points at random, and hence fully automate segmentation of the image into various tissues: scalp, CSF, gray matter, and white matter.

The MRI based DeCarli Patent utilizes measurements of volume to define regions of interest (ROI) based on histogram intensity of threshold-defined structures. The DiCarli patent teaches determination and identification of disease presence by searching for differences in volume and teaches monitoring of disease progression by observing volumetric changes. The DiCarli patent can be used therefore to determine volumes of various regions in the brain and to detect AD and other disorders that may lead to volumetric changes occurring at later stages of the disease.

This may be distinguished from the instant PET based patent which uses data from the measurements of glucose metabolism to define three dimensional spherical volumes of interest (VOI) of 1 cm diameter where the center of the sphere is located by using a mathematical treatment based on statistical parametric mapping (SPM). The 1 cm diameter, spherical VOI is roughly equivalent to a cube of 125 volume elements (voxels); i.e. a cube 5 voxels on a side where each voxel is a 2 mm isotropic volume element.

Statistical parametric maps are spatially extended statistical processes that are used to test hypotheses about regionally specific effects in neuroimaging data. The most established sorts of statistical parametric maps are based on linear models, for example analysis of covariance (ANCOVA), correlation coefficients and t-tests. Application of SPM brings together two well established bodies of theory (the general linear model and the theory of Gaussian Fields) to provide a complete and simple framework for the analysis of imaging data. SPM is a software package that consists of a collection of tools used to process and analyze 3D functional brain image data. SPM runs in a Matlab (Mathworks, Inc) shell. The homepage can be found at http://www.fil.ion.ucl.ac.uk/spm/. SPM is used to spatially normalize and spatially filter the brain image data (processing) and then to compare two groups of subjects and statistically analyze the results. These results are subsequently used to provide loci for sampling with MARSBAR, a “plug-in” accessory program for SPM, which actually does the intensity sampling of the 5 mm radius spherical volumes of interest. The 3D matrix calculations used for processing and analysis of PET brain image data are well documented in the art.

The DeCarli methodology has been replaced by voxel based morphometric (VBM) measurements. VBM enables whole brain analyses on segmented images, and can more precisely define where tissue loss is occurring. This technology, using MRI, holds the potential for early discrimination; however, the problem with this method lies in the inherent variance in hippocampal volume. Because of this, successive measurements are required. Thus, two scans taken at least one year apart to get two volume measurements are needed to show a downward direction in volume greater than that seen in normal aging.

One feature of the instant patent that provides an improvement over DiCarli and the methods developed using the DiCarli approach is the sensitivity to metabolic changes over a broad range of specific regions, at one, very early time point.

The U.S. Pat. No. 6,490,472 entitled, “MRI system and method for producing an index indicative of Alzheimer's disease” by Li, et al. and the related published manuscript by Li and others (Li et al, Radiology, 225(1):253) used to aid in interpretation of the patent document refers to the use of correlations between functional MRI time-series from the hippocampus region to generate an index. This index is presented as having the potential to be used as a preclinical marker for AD. In Li's method, the subject receives an MRI. Two specific pulse sequences are completed: one collects a high-resolution structural MRI image, the other collects a series of echo-planar images (one every 2 seconds) for a total of six minutes, thus a total of 180 scans. The “time-series” is simply the intensity value for a single voxel in one scan, looked at over time (number of scans). Thus, a time-series in this case would have 180 intensity values. This is processed, and cross-correlated with other time-series. Multiple time-series are collected from a region encompassing the hippocampus, which is determined by drawing the region on the structural MRI, and applying it to the functional MRI scan as a mask. All the time-series in the region are cross-correlated, and the mean of those correlation coefficients represents an index named the COSLOF index.

The patent reports a study showing a separation between the COSLOF index of AD patients and controls. This is shown in FIG. 10 of the patent. However, what isn't shown is the large overlap between MCI patients and controls. This is demonstrated in FIG. 4 of the Li report in Radiology. Despite this large overlap, this patent and supporting work is still represented as a means to detect AD preclinically. The Li studies provide a COSLOF index of 1.9 as being the cutoff point for distinguishing AD. One supposition from this is that serial measurements will be required to measure change over time, since initially there may be no difference between an MCI subject and a control. One would predict that those not destined to get dementia would not have a declining COSLOF index, while the index of those that were so destined would decline.

In summary, the Li patent uses MRI to obtain measurements of blood oxygen level dependent (BOLD) effects. The ROI's are defined by a structurally-defined location in the hippocampus. Li's method determines the presence of disease using the COSLOF index based on connectivity, and monitors disease progression by following decreases in the index based on lower functional connectivity. The method may be useful to determine connectivity in the hippocampus as of means for detecting AD and perhaps MCI but may not be able to fully discriminate MCI cases.

One interesting feature mentioned in the Li manuscript is the concept of compensatory brain activity. This refers to the phenomenon wherein activity in other brain regions may change in a manner which is compensatory to the activity changes resulting directly from developing pathology. This may be the reason for the increased metabolic activity seen in the cerebellum, pons, and motor cortex in subjects with MCI observed in the research forming the foundation of the instant invention. Defining which regions are primarily affected and secondarily (compensatorily) affected presents some difficulty. The posterior cingulate cortex often has the most sensitive decreased metabolism due to its innervation from the hippocampal area. It is suggested that improvements to the index taught in Li could be improved by examining functional connectivity between the hippocampus and other structures.

The method taught in Li is restricted to the medial temporal lobe in AD patients. The findings do not extend to MCI. The method could be greatly improved by obtaining data from MCI patients, performing resting state fMRI, and doing connectivity analysis to compare and contrast various specific regions.

In contrast to the Li patent, the instant invention is based on PET measurements of glucose metabolism. The three dimensional VOI's are based, as described previously on spheres with 5 mm radii at locations defined by SPM maxima. Disease is determined by searching for differences in the CDI values. Disease progression may be monitored by observing changes in metabolism. The method in the instant patent is useful for detection of MCI as a leading indicator of AD and other disorders that cause specific regional patterns of metabolic change and which can be verified by calculating changes in the CDI.

U.S. Pat. No. 6,430,430 entitled, “Method and System for knowledge guided hyperintensity detection and volumetric measurement” by Karen Gosche relates to the use of MRI for the automated segmentation and volumetric measurement of white matter hyper-intensities, typically seen in Multiple Sclerosis (MS). Regions are defined by histogram intensity threshold-defined structures This patent bears a great deal of resemblance to the De Carli patent mentioned above, as it is based on automated segmentation and thresholding algorithms. However in this case the segmentation and volume measures go beyond individual brain structures and regions and also looks for specific and characteristic white-matter lesions found in MS. Because these lesions are hyperintense, they can be detected with relative ease by automated image segmenting and thresholding methods and their volumes subsequently measured. This could theoretically be applied to any such lesion with a significant intensity difference, either naturally or by the injection of contrast material to enhance visualization of the lesion, although this does not appear to be mentioned in this patent. The method taught in the Gosche patent to determine disease presence by searching for differences in volume and/or hyperintensity is essentially an extension and refinement of the teaching in the De Carli's patent discussed above. The Grosche patent teaches monitoring of disease progression with volumetric/intensity changes and could be useful to determine volume of various regions, detection of MS lesions and other disorders, such as AD, that induce volumetric or intensity changes. This stands in contrast to the instant patent using PET to measure metabolic changes in the previously described VOI's based on a volume radius as small or smaller than what might be seen in a MS lesion advanced enough to trigger the Gosche patent's hyperintensity threshold. The instant patent determines disease presence by identifying differences in CDI values that occur early in the disease progression and monitors disease progression by tracking metabolic changes and the concomitant changes in the CDI. The contrast of the instant application to the cited patents shows the utility of the instant invention's use to detect MCI arising from a broader class of disorders, at an earlier stage of development and, subsequently, of greater opportunity for the patient to obtain earlier, beneficial intervention.

U.S. Pat. No. 6,374,130 entitled, “Methods for tracking the progression of Alzheimer's disease identifying treatment using transgenic mice” by Eric Reiman relates to the determination of radioactive glucose (FDG) uptake in the posterior cingulate region of transgenic mice. These mice have been transfected with a human gene that increases risk for AD, and have been treated with various agents which may have potential for treatment of AD. The posterior cingulate region in the brain is recognized as a region where metabolic changes have a high degree of sensitivity in the detection of AD. Mice have metabolic decline in this area related to the onset of pathophysiological changes brought on by the transfected gene. This then becomes a model for the detection of agents that have the potential to treat AD, in that an agent which prevents or slows down the metabolic decline in the posterior cingulate region may prevent or slow down AD. Said agent could therefore be used in clinical trials in humans.

In summary, the Reiman patent teaches a use of autoradiography to measure glucose metabolism in the posterior cingulate region of the mouse brain. Disease presence is determined by assessing metabolic decline. The method taught in the Reiman patent may be used to monitor disease progression upon observation of glucose metabolism changes in the posterior cingulate region. This model may be useful for assessing efficacy of potential therapeutic agents applied to treat AD or other conditions.

In contrast, the instant patent utilizes PET measurements of glucose metabolism in a plurality of three-dimensional volumes determined by SPM maxima-defined locations. The SPM techniques are thus applied more broadly and reveal those volumes exhibiting the greatest intensity change. The method in the instant patent is not restricted to only one region as is the Reiman patent. The method taught in the instant patent may be used to monitor disease progression by searching for differences in CDI values. These changes, subtle in the onset of disease, are detected at an earlier stage in humans and may be correlated with psychological and other tests of mental acuity. The methods of the instant patent are, therefore, useful for detecting the changes in MCI resulting from the onset of AD, other degenerative diseases or injury to the brain. While the posterior cingulate region is one of the most sensitive to the degradative onslaught of AD or similar diseases, it is not the only region that shows change. Further, the instant method may be applied to determine MCI in closed head injury causing trauma to other regions of the brain than are customarily investigated in degenerative disease. Given the variation in the human population, the SPM overlay used to identify regions of maximal change is clearly superior.

The U.S. Pat. No. 5,632,276 and its continuation, U.S. Pat. No. 5,873,823 entitled, “Markers for use in screening patients for nervous system dysfunction and a method and apparatus for using same” by David Eidelberg, et al. address the use of PET measurements of glucose metabolism in Parkinson's disease (PD). The patents recite possible application of the methods in AD, but include no description of any work done with patients suffering from AD. These patents teach use of a Scaled Sub-profile Model (SSM)

Pat. Nos. 5,873,823 and 5,632,276 (with the below cited references, collectively, Eidelberg) describe how PET brain image data is obtained and stored digitally on a computer. The image is transformed into standardized stereotactic space by what is described as a “resizing and reorienting” procedure. The processed image is then spatially filtered. Patient scan images are then sampled in various regions, and these sampled data are entered into an analysis. The analysis generates a “patient profile”, which purportedly can be used to diagnose and discriminate this patient from other patient populations. The pattern of metabolic covariance (a subtype of factor analysis of variance, or FANOVA) within those regions is used to predict or indicate the presence or absence of PD. Reported sensitivity for this method is 75-95 percent. (Eidelberg et al., Early differential diagnosis of Parkinson's disease with 18F-fluomodeoxyglucose and positron emission tomography, Neurology, 45(11):1995-2004 (1995), Eidelberg et al., Assessment of disease severity in parkinsonism with 18F-fluorodeoxyglucose and PET, J. Nucl. Med. 36(3):378-83, (1995).

There are several major significant differences between the method described in the U.S. Pat. Nos. 5,873,823 and 5,632,276 patents and the instant patent. A brief comparison of these differences is presented.

Eidelberg and the instant patent both use FDG PET scans Both involve collecting PET brain image data, digitizing the images, and storing digitized images on a computer. In terms of processing of the images, both similarly use the spatial filtering methods well known in the art. Both sample data from various regions in the brain, and calculate a number(s) based on the sampled data. Both use the results to predict or diagnose neuropsychiatric illnesses.

The differences between Eidelberg and the instant invention begin with the very application of the PET scan processing. The actual steps used in the generation of an FDG PET brain image are complex. Image data can be collected in 2D mode or 3D mode, reconstruction can be one of several major different types, and attenuation can be one of several types as well. Attenuation correction is very important as the signal change through the depth of the brain can be affected, and hence signal-to-noise can be altered. The instant patent is based on data showing that both reconstruction and attenuation correction algorithms affect the sensitivity of the CDI. No mention of any image collection, reconstruction, or attenuation correction routines is made in the Eidelberg patents. Further, standardization of an image in 3D space can be done in a number of ways. There are three axes, and at least four linear warps that can take place along those axes (translation, rotation, scale, and shear). Typical routines use a least squares approach that minimizes the difference between a parent image and a target image with 12 parameters, and this is the method used in the instant patent. The “resized and reoriented” processing is not well defined in the Eidelberg patents. Resizing could mean scaling, or it could simply mean removing extraneous space outside the brain image, and translating the image to a central standard location in the image space. Reorienting refers to rotation along the three axes. Thus, it may be inferred that the Eidelberg patents likely refer to a 6-parameter transformation, but at most, 9-parameters. This step is important as the definition of volumes of interest for the CDI are based on spatially standardized (12-parameter) images, not just coregistered (6-parameter) images.

In considering the differences between Eidelberg and the instant invention in data sampling from volumes of interest, it is unclear exactly which brain regions the data is sampled from in Eidelberg. Regions described in the earlier published literature of Eidelberg do not appear to be 1 cm diameter spheres, and thus it is unclear whether these are the same regions or not, and they still are not clearly defined as to location. Regions used in the present work are clearly defined, and based on the results from objective statistical analyses as being the most significantly different across the group analysis.

The mathematical methodology that creates the profiles in Eidelberg is completely different than the one used in the creation of the CDI as taught in the instant patent. The CDI, as explained below, is a simple formula, but a prerequisite is weighting of the VOI's based on a frequency analysis, as described herein. As seen in the description of the instant invention below, the formula is defined as a ratio of the mean of four regions identified with increased metabolism to the mean of nine regions with decreased metabolism, and combined with standardizing the group grand mean to a value of one with a scale factor (normalization). While the calculation of the weights for the portions of the Cognitive Decline Index (CDI) has some slight similarity to the way that an artificial neural network is created and trained, there is essentially nothing about the two mathematical algorithms that is similar.

Clinically, the CDI is a marker that can be used to predict AD. The marker/method described in Eidelberg relates to the detection and diagnoses of multiple disease states, including both PD and AD. While it may be possible at some point in time to alter the CDI taught by the instant invention to be predictive of PD, one of the underlying differences between these two methods is that the SPM analysis provides loci that are specific for MCI, and thus the loci sampled as used in the CDI are specific for MCI and AD. For diagnoses of PD, an SPM analysis of patients with PD compared to control would have to be completed. While the same method as used for CDI may be very accurate and sensitive for the detection of PD, the current methodology is not directly translatable to that disorder.

Eidelberg uses ROI's from regions not completely defined in the Eidelberg patents. The instant invention uses VOIs based on SPM maxima-defined locations as the center of 1 cm diameter spherical (0.5 cm³) volumes. Eidelberg determines the presence of disease by assessing abnormal SSM values. The instant invention determines the presence of disease by assessing abnormal CDI values. The Eidelberg method could be utilized to monitor disease progression by observing SSM value changes. The instant invention enables monitoring of the progression of disease by following CDI changes based on changes in metabolism. Eidelberg may be useful for detection of PD, and perhaps other disorders that may cause specific patterns of metabolic change. The instant invention may be used to detect disorders that may cause specific regional patterns of metabolic change including AD, PD, alcoholism, drug abuse, and closed head injury.

The U.S. Pat. No. 5,434,050, entitled, “Labeled beta-amyloid peptide and methods of screening for Alzheimer's disease,” by John Maggio et al. (Maggio) deals with the use of a beta-amyloid peptide fragment as a label of diseased tissue. Maggio covers both in vitro and in vivo use, although all the examples are in vitro. Samples of tissue which are diseased (containing amyloid plaques) will bind the peptide fragment due to the self-binding/polymerizing nature of amyloid in plaque formation. This property is leveraged in the ability of the peptide to label plaques in a given tissue. Maggio covers radio-labeled peptides, as well as a plurality of other labeling methods. While PET is mentioned as a potential means of detection of radio-labeled peptide binding in vivo, no discussion or description of how this could be carried out is given. Maggio does not address the potential problem of the failure of the peptide to pass across the blood-brain barrier.

Where the instant invention utilizes PET, Maggio teaches the use of multiple labeling methods of the beta-amyloid peptide. The instant invention is based on defining volumes of brain tissue to be examined based on metabolic activity observed in specific volumes indicated by statistical analysis (SPM) whereas Maggio does not teach the use of any specific region. The method of the instant invention may be used to determine the presence of disease by searching for differences the mathematically derived CDI. The method in Maggio determines the presence of disease in vitro by assessing amyloid binding obtained from brain tissue examined for amyloid plaques. The instant invention does not depend on brain biopsies.

The U.S. Pat. No. 5,109,868 entitled, “Method for diagnosing senile dementia of the Alzheimer's type,” by Anthony Smith et al, teaches use of both structural (CT or MRI) and functional (SPECT) imaging methods in detecting/diagnosing AD. It uses a measure of width of the medial temporal lobe nearest to the brainstem as a marker for disease presence, defined in three different ways: 25% less than average thickness of controls, ratio of 0.75:1 (patient/controls), and/or 11.5 mm or less in size. Smith further teaches use of this measure as a marker is a cerebral blood flow deficit in the temporal-parietal cortex. This was based on a subjective clinical evaluation done by a nuclear medicine physician, grading the SPECT scans from 0 (no obvious lesion) to 3 (severe perfusion deficit crossing the cortical rim). These are rather primitive measures by today's standards. SPECT is still more commonly used than PET for detection of AD, mainly due to cost. However it should be noted that this study is for the detection of AD, not MCI. These markers are unlikely to indicate any change in MCI.

Much has changed in the intervening fourteen years between issuance of Smith in 1992 and the present. The basic methodology taught in Smith lacks objectivity, and requires arbitrary location of regions to make measurements. The use of the two methods taught in Smith to better assess disease are useful and have been employed in the art.

In summary, the methods in the Smith patent utilize CT/MRI and SPECT in contrast to the PET of the instant patent. Smith teaches measurements of structure—width of the medial temporal lobe nearest to the brainstem (CT/MRI) and cerebral blood flow (SPECT) as opposed to the measurements of metabolism of the instant patent. The single region for CT in Smith is based on a defined anatomical location (posterior medial temporal lobe) and the general locale of the temporal-parietal cortex (SPECT). The instant invention is derived from specific locations defined by SPM. Smith teaches the determination of AD by assessing decreased width of the medial temporal lobe nearest to the brainstem and decreased perfusion. The method in the instant invention contemplates determination of degenerative diseases or injury by searching for differences in CDI values and is not restricted to AD. The methods taught by Smith have the potential to monitor the later stages of AD progression by observing decline in both measures. The CDI described in the instant invention monitors progression of disease based on the change in the CDI which is likely to discern the existence of AD or other degenerative disease at an earlier stage of development.

The U.S. Pat. No. 5,617,861 entitled, “Magnetic resonance spectral analysis of the brain for diagnosis of clinical conditions,” by Ross, et al. refers to measurement of brain metabolites using nuclear magnetic resonance spectroscopy (MRS). Both MRS and MRI are performed with the same hardware. Different software components are used to achieve the separate results. The spectra involved consist of a series of peaks, as represented in Ross, which represent various chemicals: creatinine, N-acetyl aspartate, myo-inositol, and some others. These chemicals are metabolites of cell function. The essential feature of MRS to understand is that these peaks change in various ways with various disease states. To measure changes in these metabolites, one begins by identifying a particular volume element (voxel) on the brain image, and utilizes the software to collect data from that region. A voxel as defined for MRS is quite different than a voxel (elemental volume element in a 3D image) used elsewhere. The MRS voxel is essentially a region of interest. This typically encompasses 10 cm³, a rather large volume of brain tissue. The size of the voxel is directly related to the amount of time the subject being tested must be scanned to obtain spectra of a given quality.

Developments in the art following Ross recite use of Ross' methods. However, it appears that the structurally based or morphometric approaches reviewed above develop more sensitivity in actually indicating MCI.

In contrast to the method of the instant invention measuring glucose metabolism, the Ross method teaches measurement of specific brain metabolites, myo-inositol, creatine and N-acetylaspartate. In Ross, the volume in which the metabolites are measured may be as large as 10 cm³ in the medial temporal lobe or the posterior cingulate region. This is in contrast to a volume of 0.51 cm³ in SPM maxima-defined locations in the instant patent. The larger volume in Ross is required by the mechanics of the measurement process rather than by the localization of precisely where in the region being examined the degenerative process causing cognitive decline occurs. In fact, the use of the larger volume in Ross, while it may include the diseased region, tends to diminish the sensitivity of the technique because the values of the metabolite changes in the diseased region are averaged in with the more normal values obtained over the remainder of the voxel.

The method taught in Ross was used to evaluate only several, subjectively chosen regions of the cortex. The data used to substantiate the Ross method shows a significant degree of overlap in values between the subject determined to have MCI and control populations.

For the foregoing reasons, there is a need for a non-invasive, early stage method to obtain quantitative measures of mild cognitive impairment useful in diagnosing and following degenerative brain disease or closed head injuries. The methods taught herein address this need by using in situ analysis of glucose metabolism in the brain using positron emission tomography, analyzing and transforming the image data and using the data to construct a cognitive decline index (CDI) measure mild cognitive impairment (MCI) indicative of the consequence of degenerative brain diseases or traumatic, closed head injuries.

Additional commentary regarding these and other studies is provided in the Detailed Description of the Invention below that incorporates more detailed references the teachings of the instant invention.

### SUMMARY OF THE INVENTION

Clinical diagnosis of degenerative brain diseases such as Alzheimer's Disease, Parkinson's Disease and related disorders is currently imprecise. There have been, to date, no biochemical indicia that relate early stage deterioration and accompanying cognitive impairment and which can be used to identify these diseases and quantify the findings related to treatment. Similarly, head trauma resulting in closed head injury with concomitant cognitive impairment lacks quantitative diagnostic methods.

This method enables the early detection of Mild Cognitive Impairment, a prodrome to Alzheimer's Disease. The methodological components include the specific location of the brain volumes of interest (VOIs) along with specific weighting factors derived from comparison of patient data and normal controls, and the creation of the normalized CDI from the mean weighted VOIs. Spatial normalization and filtering of a given image can be coded simply without having to use Matlab or SPM, and a one cm diameter spherical VOI at each coordinate can be sampled without the use of Marsbar, as these are fairly straightforward mathematical algorithms. Results of an exemplar study used to establish the preferred embodiment of the instant invention are presented here in a group comparison format, but the methodology taught is applicable to develop a CDI for and to evaluate a single patient. The preferred embodiment of the instant invention comprises a set of software routines that can independently provide a CDI value for an appropriately processed FDG PET scan of a patient's brain. In addition to these considerations, the high sensitivity of the CDI enables its use as a screening tool for the early detection of a variety of cognitive disorders.

Data from experiments performed to develop the instant invention show that analyses of groups of premorbid individuals can be discriminated from groups of normal controls To date, there is no reliable method of detecting MCI in the early stages, at the clinical level, in individual patients. The development of the methods taught in the instant invention are overdue for clinical use to put this early detection capability into the hands of clinicians. Detection of the degenerative processes in premorbid or very early stages of brain degenerative disease, drug abuse or immediately following brain injury would enable the early treatment with medication to enhance and prolong quality of life, to provide an answer to the patients' questions regarding their potential for cognitive decline, to help them plan and prepare for the future, and hopefully one day, to prevent these diseases altogether or provide increasingly effective rehabilitative measures for injury.

It is therefore the object of the invention to address the need for a non-invasive, early stage method to obtain quantitative measures of mild cognitive impairment useful in diagnosing and following degenerative brain disease or closed head injuries by utilizing the image data from individual patient positron emission tomographic scans to construct a cognitive decline index which can serve as a diagnostic and screening tool to reveal the onset of mild cognitive impairment and nervous system dysfunction which are sequelae of degenerative brain diseases and closed head injury.

It is a further object of the invention to provide a method for determining the severity of said brain diseases or injuries.

It is a further object of the invention to use successive measurements over a period of time to track the progression of degenerative brain disease in individual patients.

Still another object of the invention is to provide a method for producing an index indicative of brain disease comprising the steps of collecting positron emission tomographic image data showing metabolic activity in the brain of a patient, spatially normalizing said image data using a standardized three dimensional coordinate system, and spatially filtering the normalized image data. Specific regions of the brain showing extremes in metabolic activity are selected and mean intensity values are collected for the normalized, filtered image data from said selected specific brain regions. The mean intensity values are weighted with standard weights derived from the group analysis used to create the standard and the ratio of the mean, weighted, metabolic activity image data are normalized to produce a numerical index.

It is a further object of the invention to assemble and maintain a data base of findings from individual patients which can provide reference points for comparison in the ongoing effort to understand and treat these diseases and conditions.

It is a further object of the invention to monitor the effect of treatment of brain diseases and injury to quantify the effect of said treatment in ameliorating the disease or injury.

In one embodiment of the invention, the method may be used for diagnosing Alzheimer's Disease at an early stage by measuring functional activity in a patient's brain and using the method to quantify mild cognitive impairment by constructing a cognitive decline index. The CDI may be correlated with other measures of mental acuity.

In another embodiment of the invention, the method may be used for diagnosing Parkinson's Disease by measuring functional activity in a patient's brain and using the method to quantify mild cognitive impairment by constructing a cognitive decline index. The CDI may be correlated with other measures of mental acuity.

In another embodiment of the invention, the method may be used for determining the severity of cognitive impairment by measuring functional activity in a patient's brain, using the method of the instant invention to construct a cognitive decline index and correlating the index with other measures of mental acuity.

These and other features and advantages of embodiments of the instant invention will become better understood with reference to the following description, appended claims and accompanying drawings.

### BRIEF DESCRIPTION OF THE DRAWINGS

FIG. 1 is a flow chart of the steps utilized in composing a database of normal control subjects and selection of patients for the application of the Cognitive Decline Index (CDI) FIGS. 2A, 2B, and 2C are flow charts of the steps required to practice the preferred embodiment of the instant invention.

FIG. 3 shows FDG PET scans of a normal subject control subject and a patient with Alzheimer's disease depicting enlarged regions of hypo-metabolism in the bilateral parietal and posterior cingulate region in the patient's brain as well as a more prominent motor strip as compared with the normal subject.

FIG. 4 depicts Statistical Parametric Mapping (SPM) results showing decreases in brain metabolism in patients with early cognitive impairment compared to controls in a group analysis. Results are displayed in neurological orientation (images left is subject's left). The top left display is a maximum-intensity projection (MIP) image, AKA “glass-brain” image. This display shows all the voxels that were significant at the threshold chosen for display. For the purposes of this display, that threshold was fixed at a T of 2.5, and cluster size of 50 voxels. The upper-right shows the design matrix for the SPM compare-groups analysis. Below that are the read-outs for coordinates, cluster-level, and voxel-level statistics. The most stringent is voxel-level (corrected), and the most relaxed is voxel-level (uncorrected). Below that are some descriptive parameters for the statistical analysis, and below that is another table of coordinates from the large cluster in the left parieto-temporal area. The main readout from the results (the table above) will only report the three main foci in a blob. If there is a large blob, as is the case with the one in question, it is possible to get a printout of all the maxima in that blob, as was done here.

FIG. 5 depicts SPM results showing increases in brain metabolism in patients with early cognitive impairment.

FIGS. 6A and 6B depict a cross-section (a) and 3D rendering (b) of regions of metabolic decrease in brain metabolism in patients with mild cognitive impairment compared to the subset of normal controls. The numeric color scale represents the SPM(t) values.

FIGS. 7A and 7B depicts a cross-section (a) and 3D rendering (b) of regions of metabolic increase in brain metabolism in patients with mild cognitive impairment compared to normal controls. The numeric color scale represents the SPM(t) values.

FIG. 8 shows (a) a frontal, (b) lateral and (c) coronal views of the images from a FDG PET scan with the size and location of a volume of interest (VOI) superimposed.

FIG. 9 displays results for the CDI₁ across four groups of subjects (controls mild cognitive impairment patients from a pilot study, mild cognitive impairment patients identified retrospectively and Alzheimer's patients identified retrospectively) in the experiment reported in reducing the preferred embodiment of the instant invention to practice.

FIG. 10 shows the correlation of age vs. the CDI₁ for the four groups of subjects recited in FIG. 9.

FIG. 11 shows the separation obtained when using externally determined weights and examining only the MCI patients and older controls.

FIG. 12 shows the distinct separation obtained when comparing MCI patients to older controls using externally determined weights as they relate to age.

### DETAILED DESCRIPTION OF THE PREFERRED EMBODIMENTS

The data, analysis, calculations and procedures forming the preferred embodiment were produced in studies of four groups of patients:
1. Control subjects from the normal control database,
2. Patients identified retrospectively with early cognitive decline who received negative PET workups,
3. Patients identified retrospectively with cognitive decline and with the pathognomonic changes of AD present on PET imaging,
4. Patients with MCI from the pilot study.
   The data and statistics used as exemplars in the figures and tables are taken from these studies.

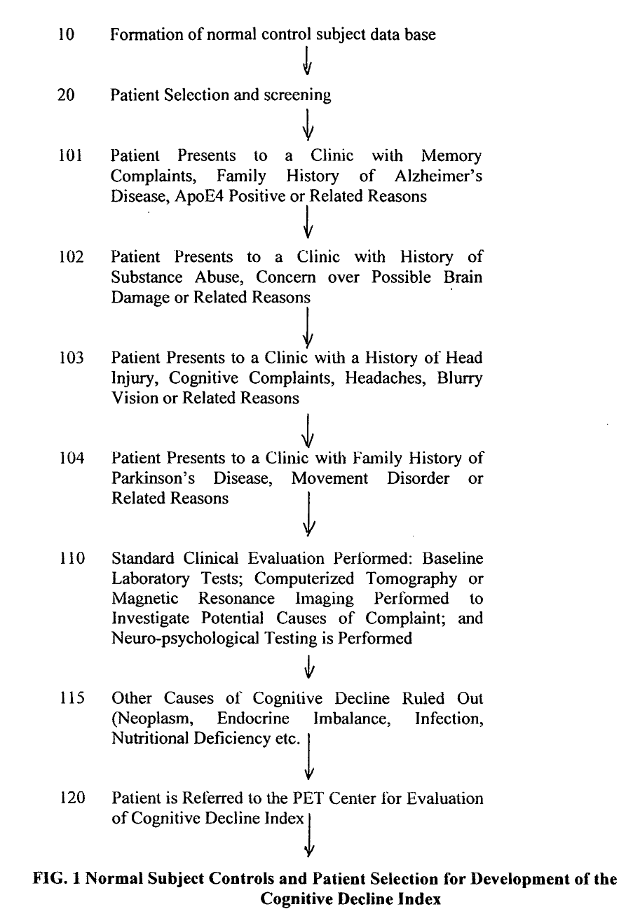

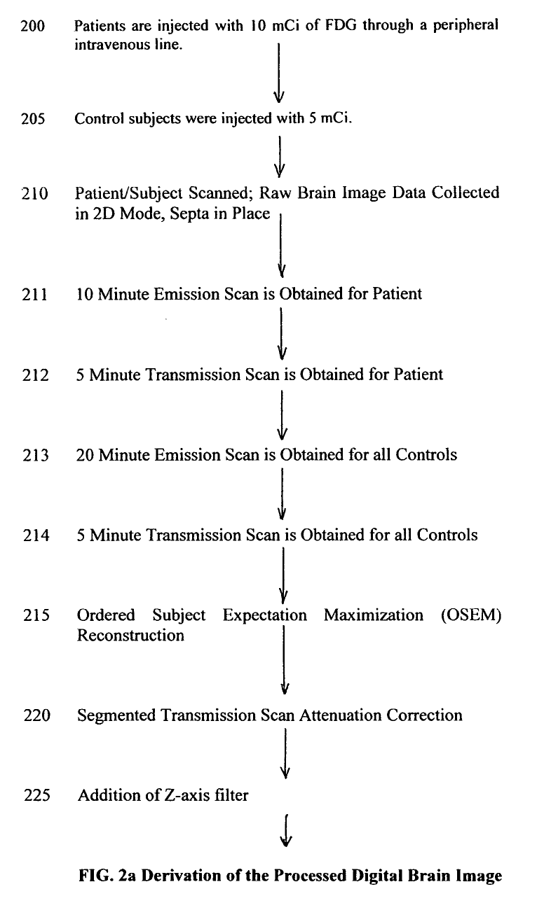

Referring first to FIG. 1, a database 10 is compiled for normal control subjects.

The database of FDG brain scans from healthy control subjects was created to enable the objective examination of a variety of patients who presented for clinical evaluation of cerebral pathology. These subjects are physically examined and screened for neurological and psychiatric illness. Entry into the data base requires that subjects have normal or unremarkable MRI scans as well as negative cognitive tests on Folstein Mini Mental Status Examination (MMSE)>28 (Folstein et al., 1975). The control subjects' data from FDG PET normal brain scans is validated by comparison with patients with cerebral lesions. The normal control subject database is then used for statistical comparison in the method described as part of the instant invention.

Referring to FIG. 1, patients are selected and screened 20. Patients are categorized by the symptoms and history presented at examination. There are four categories: 101 memory complaints, family history of Alzheimer's disease, ApoE4 positive or related reasons; 102 history of substance abuse, concern over possible brain damage or related reasons; 103 history of head injury, cognitive complaints, headaches, blurry vision or related reasons; and 104 family history of Parkinson's disease, movement disorder or related reasons. Standard Clinical Evaluation is performed on the presenting patient 110 including physical examination, baseline laboratory tests; computerized tomography or Magnetic Resonance Imaging and neuro-psychological testing including the MMSE as well as the clock-drawing test (Shulman et al., 1986, Kirby et al, 2001) to search for memory and visuo-spatial impairments. These tests were chosen based on established sensitivity demonstrated in the literature (Petersen et al., 2001, Chen et al., 2001).

Other causes of cognitive decline are ruled out 115. These include neoplasm, endocrine imbalance, infection, nutritional deficiency etc. The patient is referred to the PET Center for evaluation of Cognitive Decline Index 120. Referring to FIG. 2a, the CDI is designed to work with FDG PET brain scans obtained from a patient in standard clinical fashion. Standard clinical FDG PET scanning procedures were employed. Patients are injected with 10 mCi of FDG through a peripheral intravenous line. 200 The patient rests quietly in a darkened room with eyes and ears open for one hour for tracer uptake before scanning is begun. Control subjects were injected with 5 mCi 205 to decrease radioactive exposure, and the scan time for the emission scan is subsequently increased to 20 min. 213 to obtain equivalent counts.

Scanning is carried out on a GE Advance PET scanner in 2D mode with septa in place for all scans as at step 210. A 10-minute emission scan is obtained 211 (20 min for controls 213), as well as a 5-min transmission scan 211, 214. CDI sensitivity changes with variation in processing methods, and is optimized using reconstruction based on ordered subset expectation maximization (OSEM) 215 and segmented transmission scan attenuation correction (SAC) 220. In addition to standard clinical processing, a z-axis filtering step is added here as well 225, to improve attenuation correction in the cerebrum and cerebellum on the base of the brain images are then reformatted for clinical evaluation on the GE Advance Workstation (Sun™ Ultra 60) 230, and a copy prepared for Export to the research workstation for use with SPM 235 (Friston et al, 1995a,b).

On the research workstation, scans are converted to Analyze 7.5 ™ (Analyze Direct, Lenexa, Kans.) format 240. Initial image voxel resolution was 3.5×3.5×4.5 mm. These images are then further processed and analyzed with SPM99 (SPM, Friston et al, 1995a) implemented in Matlab (Mathworks, Natick., Mass.) 245.

Facility in management of the image data is achieved by utilizing the Digital Imaging and Communication in Medicine (DICOM) format. 250 (see, http://medical.nema.org). The (DICOM) standard was created by the National Electrical Manufacturers Association (NEMA) to aid the distribution and viewing of medical images, such as CT scans, MRIs, and ultrasound. Additionally, the image data may be further conditioned to enable data management on particular work stations. 260 and 265

Once the image is loaded into SPM, it is spatially transformed based on a brain template into the standardized 3D space developed by the Montreal Neurological Institute (MNI). 270 There is a large amount of normal variation in size and shape of the human brain and standardization is necessary to compare patients to the normal controls as well as achieving comparability between patients for the patient database. The standardization system and its resulting coordinate space is known as Talairach space, and has been previously described (Talairach and Tournoux, 1988). Coordinates are transformed using mni2tal (Matthew Brett's mni2tal.m can be found at http://www.mrc-cbu.cam.ac.uk/Imaging/mnispace.html).

The method for standardization of image data to this space (spatial normalization) involves the use of twelve-parameter linear affine mathematical routines to translate, rotate, scale, and shear the image along the X, Y, and Z axes (4 actions×3 axes=12 parameters). A template brain scan is used as a standard, and the brain scan being normalized is matched as closely as possible to the template in shape, size, and space. This has been well described previously (Friston et al, 1995a). Briefly, the brain image data is moved (translated) to the center of the image, twisted to match the orientation of the template (rotation), and scaled to best match the size of the template. The fourth step involves shear, such that one plane of the image slides on the next to optimally fit the shape of the object image to that of the target (template) image. All four steps are carried out along all three axes in 3D space. A final processing step 280 to optimize across-subject analyses of PET image data is spatially transformed spatial filtering, also known as smoothing. This step increases the signal-to-noise ratio and decreases variance across the PET image data by removing much of the variability between patients due to differences in gyri/sulci patterns. This is necessary to achieve optimal comparability in the patient data base. The optimal smoothing level is generally agreed in the art to be approximately 1.5 to 2 times the full-width half-maximum parameter of the scanner, considered to be the spatial resolution. For this research, the GE Advance scanner has a resolution of approximately 3.5 mm×3.5 mm×4.5 mm. Spatial filtering with a Gaussian kernel (the standard method) also renders the data amenable to analysis via methods incorporating the theory of Gaussian fields, which is important for the majority of the statistical routines used by the SPM software package.

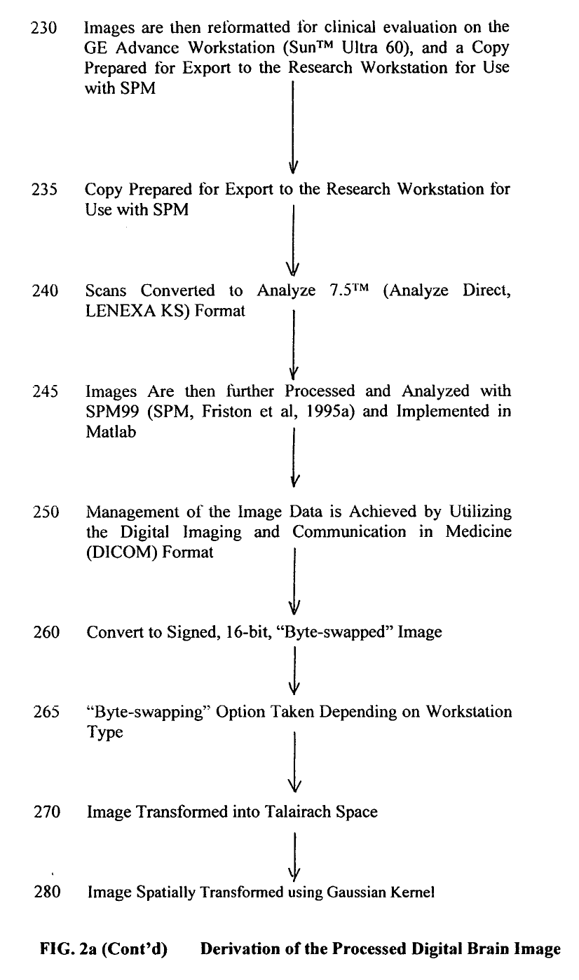

The PET scanner measures the energy from positron emission, and an image of brain function is created for the entire brain. An example is shown in FIG. 3, with the image in the top panel (a.) from a 65 year-old normal subject, and (b.) is an FDG brain image from a 67 year-old patient with probable Alzheimer's dementia. This pattern is pathognomonic of AD. However the gold standard is still brain biopsy, with tissue diagnosis based on amyloid plaques and neurofibrillary tangles. It is notable that often the FDG brain scans from patients with MCI appear normal, just like the scan in (a.), even to the well-trained nuclear medicine physician's eye. Thus, these scans are read clinically (subjectively) as not consistent with MCI or AD. Radiological evaluation of the MRI is even less sensitive, and often patients with moderate AD, like the one in (b.) still have normal or clinically unremarkable MRIs.

The two brain scans in FIG. 3a and 3b are examples of scans that have been spatially normalized. This spatial transformation can be problematic in images that have large lesions or severe atrophy, however in patients with lesions too subtle to detect clinically and/or mild atrophy there is very little error. Nevertheless, all brain images were inspected visually post-normalization to assess the quality of the transformation. One notable change from the standard is that all images were interpolated into the template spatial bounding box, instead of the standard bounding box. The template boundaries include the entire brain including the cerebellum, while the standard one cuts out the majority of this important brain component. After the spatial transformation, further residual inter-subject differences (due mainly to variation in patterns of gyri and sulci) were minimized by smoothing with a Gaussian filter kernel (8 mm isotropic). This also serves the purpose of ensuring the data are normally distributed; hence Gaussian Field theory can be applied in the analysis of the images (see below). No partial volume correction was carried out for any scans in either group, and there were some scans in both groups with mild generalized atrophy. A previous study examines partial volume correction as a means to correct for the effects of atrophy on the PET metabolic data (Meltzer et al, 1996). However, we felt this was unnecessary as there were very few subjects with clinically detectable atrophy, the atrophy present was mild, and there were both MCI patients and controls with mild atrophy. Further, another report finds that atrophy does not play a major role in PET metabolic data (Ibanez et al, 1998). Final image voxel size was 2×2×2 mm. These spatially normalized and smoothed images were then used in the SPM analyses to determine regions of significant difference in metabolism between the controls and patients.

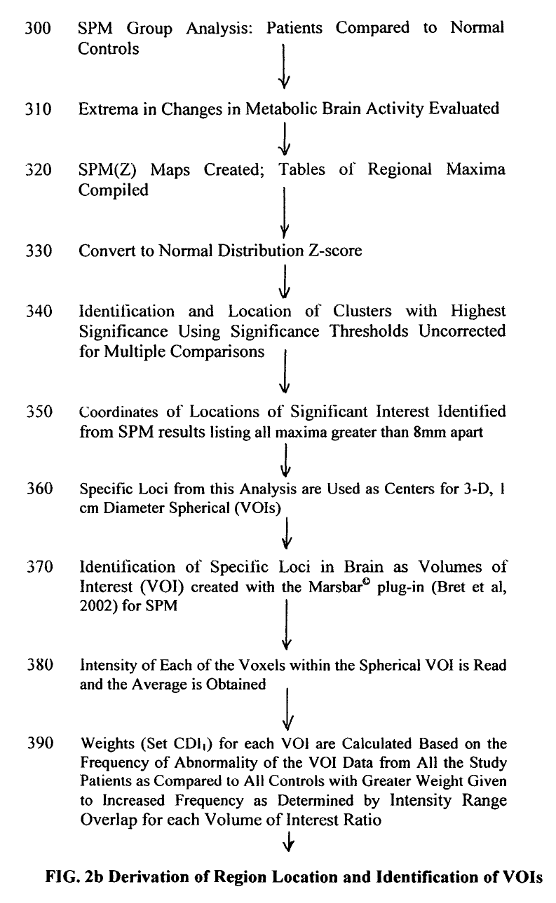

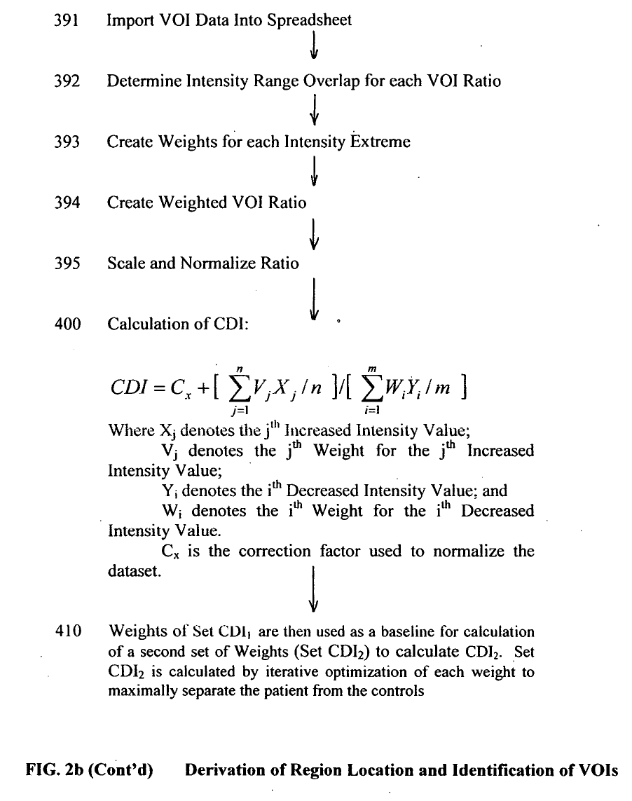

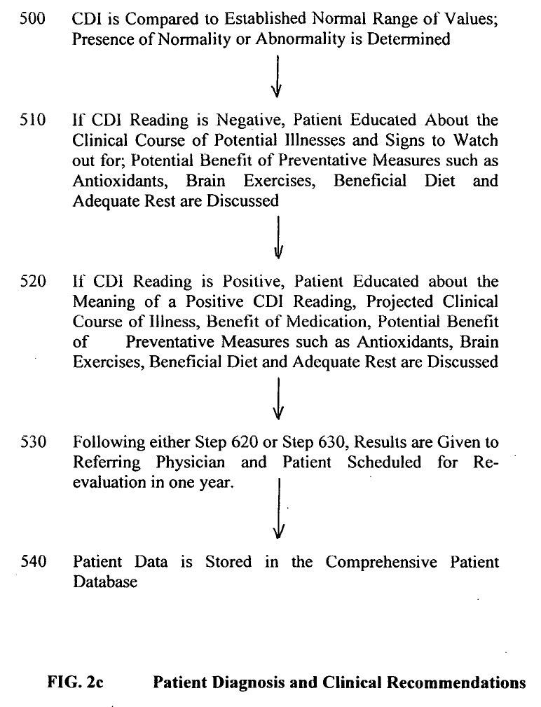

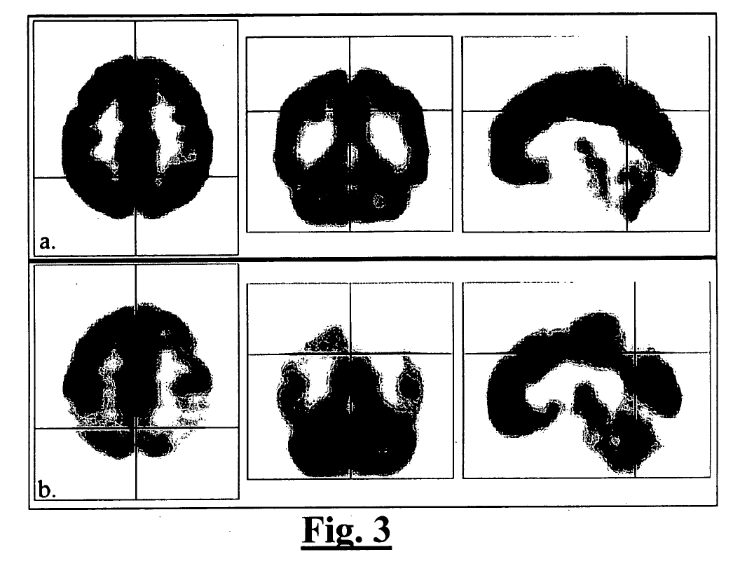

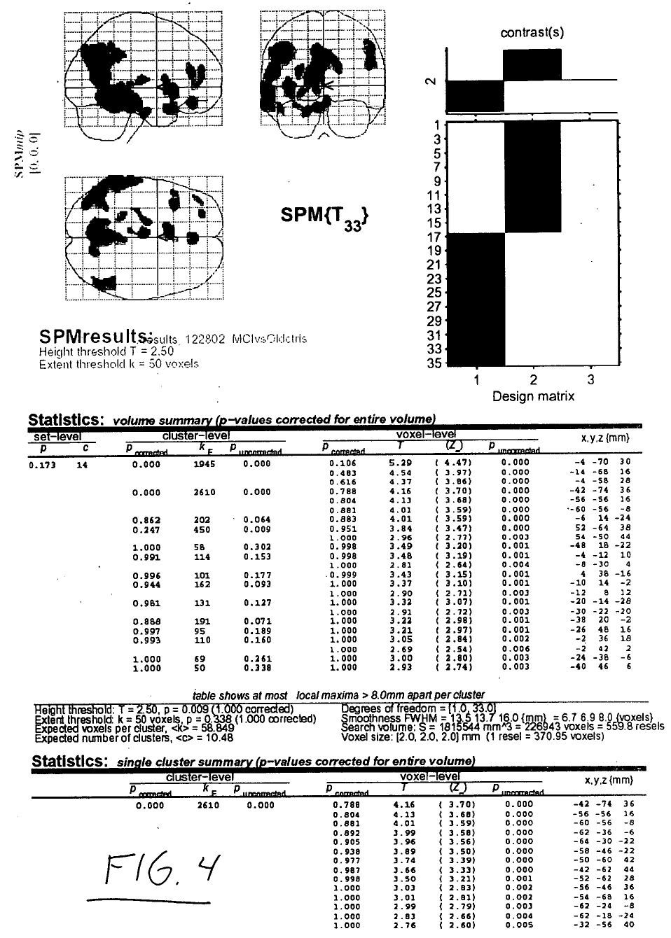

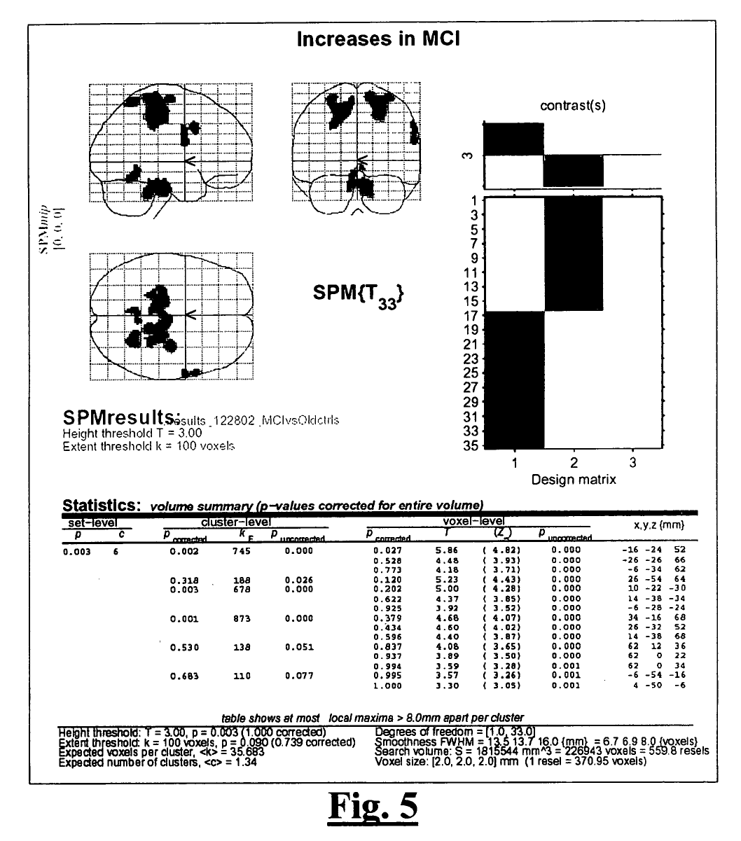

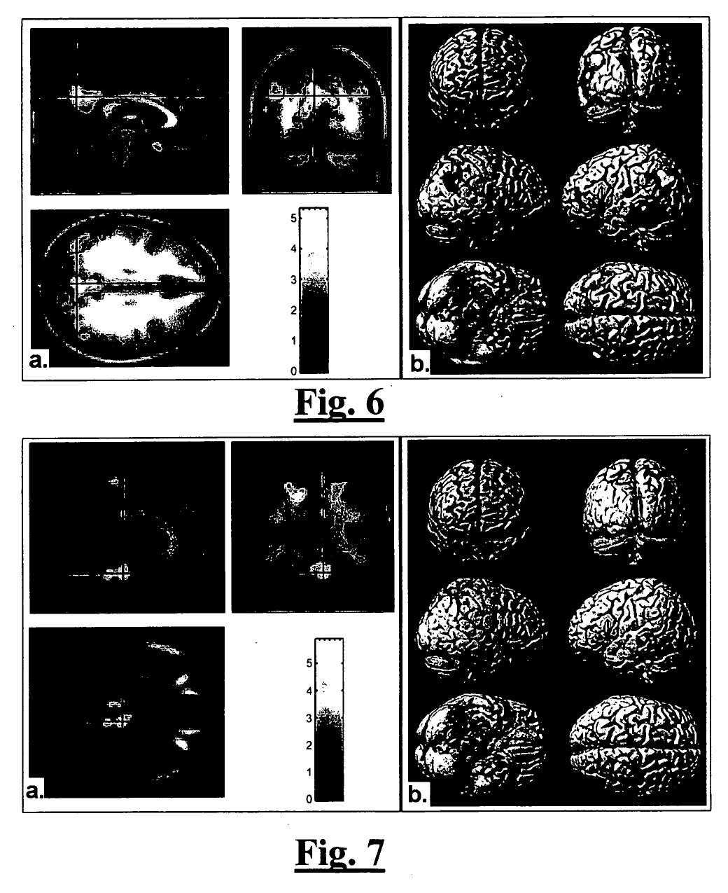

Referring again to FIG. 2b, the patient data is compared to controls of a similar age range 300 to identify extrema in the increases and decreases in metabolic activity 310. The group analysis was carried out in SPM. SPM is a software package designed for the processing and analysis of brain images. The mathematical requirements for the analyses of 3D images involve application of the General Linear Model and Gaussian Field Theory (Friston et al, 1995b; Worsley et al, 1995). The compare-groups statistical model is used in this analysis to produce a SPM(t) statistical map 320. This is then converted to the unit normal distribution Z score 330. Clusters with significant increased or decreased metabolic activity are then identified 340. A significance threshold uncorrected for multiple comparisons is used. This is supported by previous reports in the literature which indicated similar patterns of activity (De Leon et al, 2001; Reiman et al, 2001; Small et al, 2000) and by the analysis showing that many of the brain regions under examination were identified a priori as being likely regions to have decreased metabolism. This analysis enables identification of brain regions for use in creation of the CDI. The locations of significant points of interest are determined from the SPM results 350 (see FIGS. 4 and 5 for examples of the features providing these results). This printout of SPM results lists all maxima greater than 8 mm apart. Coordinates from the main foci are used in the creation of VOIs 360 for the calculation of the CDI (see below). FIGS. 6 and 7 show the maps of significant differences overlaid on canonical brain images, in both cross-sectional (a) and 3D rendered (b) views. FIG. 6 shows regions of decreased metabolism, and FIG. 7 shows regions of increased metabolism.

Specific loci from this analysis are used as centers for 3-D, 1 cm diameter spherical (VOIs) created with the MARSBAR™ plug-in (Bret et al, 2002) for SPM 370. This size VOI is selected because it approximates the spatial resolution of the data post-smoothing. Those skilled in the art will recognize, however, that other volumes may be appropriate for use as the volume of interest depending upon such factors as scanner resolution, patient tolerance of radio ligand, refinement of the statistical methods, size of both patient and normal subject database (the latter for comparison purposes as set out below), suspected disease/impairment, and other applicable factors. The intensity of each of the voxels within the spherical VOI is read and the average is obtained 380. Raw data uncorrected for global intensity differences are used, since a ratio created from these data intrinsically corrects for differences across subjects.

In the studies used to reduce the preferred embodiment to practice, mean image intensity values were collected from 13 regions for each subject. These regions are composed of areas that showed either increased metabolism (cerebellum, pons, sensorimotor) or decreased metabolism (temporal lobe, hippocampus, parietal lobe, frontal lobe, posterior cingulate). Two steps for determination of weights for each VOI are used. The first set of weights for each VOI are based on the frequency of abnormality of the VOI data from all the study patients as compared to all controls, with higher weights applied for increasing frequency 390. The CDI calculated with these weights is designated CDI₁. These weights are then used as a baseline for calculation of a second CDI (CDI₂) involving iterative optimization of each weight to maximally separate the patient from the controls according to observer criteria 395. For both CDI₁ and CDI₂ steps, the global mean of the weighted control group VOI ratio is normalized to a value of 1, and this normalization factor then universally applied. The resulting, mean, weighted, normalized VOI ratio forms the CDI.

Once CDI values were obtained for each patient in the exemplar study, the normal distribution of the data was established with a Kolmogorov-Smirnov statistic. Groups were analyzed for significant differences with analysis of variance, and the confidence level was determined for each group. Two-tailed t-tests were applied to establish the significant differences between the groups of subjects. The analysis performed in the exemplar study determined weights for each VOI. With the method completed and validated, the weights thus produced are used for each new patient that presents.

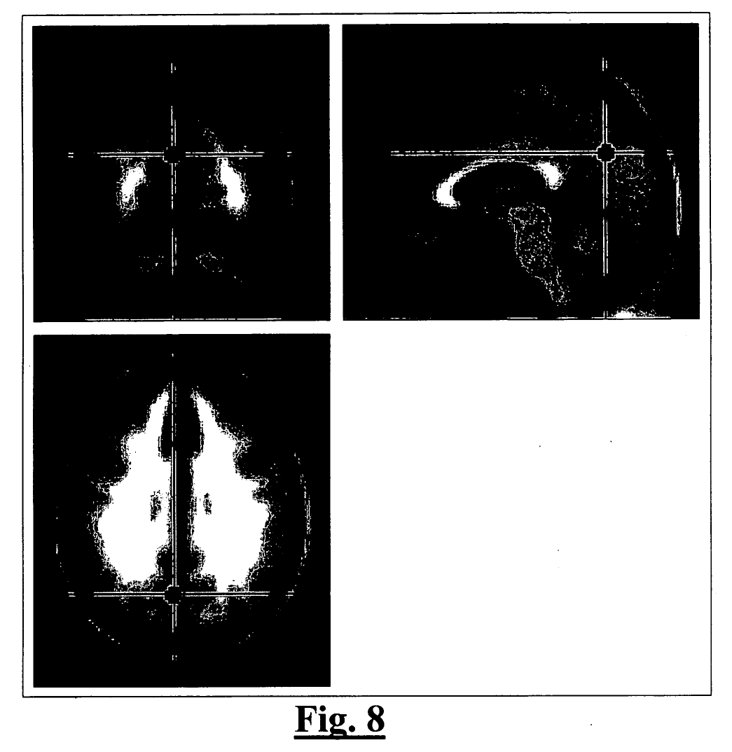

The MARSBAR™ SPM toolbox is a plug-in type program for SPM, and is used to create the 3D, one cm diameter, spherical volumes of interest (VOIs) used to sample data (Brett et al, 2002). It produces a mean intensity value for the volume elements (voxels) present within the volume of interest. The voxels are cubes 2 mm on a side after spatial normalization and the spherical VOIs represent a bounded volume containing the voxel. A VOI is thus not a perfect sphere. See FIG. 8, step 395, for an example of the location and representative size of an exemplar VOI. The CDI is derived from 13 VOIs located at specific coordinates. These include specific locations in the parietal cortex, medial and lateral temporal areas, frontal cortex, posterior cingulate cortex, as well as the sensorimotor cortex, cerebellum, and pons. In the performed study there were initially more VOIs from the majority of the maxima presented in the tables from FIGS. 4 and 5, however it was discovered that several of the regions were not necessary. This was discovered by iteratively examining the CDI results with fewer and fewer VOIs. The use of 13 VOIs proved optimal, although 11 worked well too. The sensitivity, as judged by the separation of the patients from the control CDIs, dropped somewhat if fewer regions were used, and did not appreciably increase if more regions were used. Their exact locations in 3D space are given in Table 1:

TABLE 1

| | | | |
|---|---|---|---|
| | XYZ | CDI₁ | CDI₂ |
| Region | Coordinates | Weights | Weights |

Increased activity (numerator)

| | | | |
|---|---|---|---|
| R pons | 10 −22 −26 | 10 | 20.1 |
| L vermis | −6 −54 −14 | 1 | 11.6 |
| R cerebellar nuclei | 14 −38 −34 | 7 | 18.8 |
| L sensorimotor | −16 −24 52 | 7 | 30 |

Decreased activity (denominator)

| | | | |
|---|---|---|---|
| L post. cingulate | −4 −70 30 | 13 | 5.5 |
| L frontal | −26 48 16 | 13 | 5.7 |
| L parietal | −42 −74 36 | 10 | 4.3 |
| 1^{st} L temporal | −56 −44 −20 | 4 | 4.7 |
| 2^{nd} L temporal | −56 −56 16 | 8 | 1.8 |
| L med temporal | −24 −12 −28 | 1 | 1 |
| R parietal | 54 −66 32 | 7 | −2.5 |
| Basal nucleus | −6 14 −20 | 5 | −4 |
| L post.hippocampus | −26 −36 −8 | 5 | 3 |

Weighting factors and coordinates of regions used for development of the CDI. CDI₁ weights were derived as described in the test from examination of the frequency of abnormalities in the patient group, while CDI₂weights were derived arbitrarily to optimize the separation between the two groups. This is why the image must be spatially normalized, to ensure that the VOIs are sampled at exact coordinates determined by the SPM analysis to be sensitive to the metabolic changes of MCI, in the same spot in every subject. Mean image intensity data was sampled for all 13 VOIs in all subjects. Data for each VOI and patient is displayed in MATLAB and saved to a text file for further processing and analysis. Uncorrected (raw) image intensity data was sampled, because any global intensity correction or scaling at this point is unnecessary. This is because a ratio of some VOIs to others from the same image will be obtained, and this automatically and intrinsically corrects for global inter-subject intensity differences. These VOIs formed the raw data for the creation of the CDI.

The VOI ratio without weights has good performance in separating patients from controls. This ratio is determined by obtaining the mean, \overline{X}, of the four VOIs from regions with increased metabolism (defined here as X₁ through X₄), the mean, \overline{Y}, of the nine VOIs from regions with decreased metabolism (Y₁ through Y₉) and dividing the mean of increases by the mean of decreases, \overline{X}/\overline{Y}. The results of this calculation for the above referenced study are shown in Table 2:

TABLE 2
Results from analysis of VOI ratios, without weighting or normalizing.
Note overlap between Controls and MCI.

| | Controls | MCI | AD |
|---|---|---|---|
| Mean | 0.639 | 0.779 | 0.928 |
| SD | 0.034 | 0.063 | 0.127 |
| Minimum | 0.577 | 0.697 | 0.709 |
| Maximum | 0.713 | 0.915 | 1.185 |
| Count | 33 | 17 | 15 |
| 95% CL | 0.012 | 0.032 | 0.070 |

However, the preferred embodiment is a more sensitive indicator and discriminator. The preferred embodiment is obtained by determining and applying weighting factors to each VOI. Multiple mechanisms were evaluated for determining and assessing appropriate weights. In the initial method, weights for each VOI were based on frequency of abnormality of that VOI across all patients and all controls, with higher weights applied for increasing frequency. The CDI calculated with these weights is designated CDI₁. For the frequency analysis, the un-weighted VOI values are used to generate two VOI ratio datasets. The first dataset is composed of nine VOI ratios formed by dividing the mean of the four increases, \overline{X}, by each of the decreases; namely, R₁ =\overline{X}/Y₁ for 1≦i≦9. The second dataset is composed of 4 VOI ratios formed by dividing each of the increases by the mean of the decreases; namely Rⱼ=Xⱼ/\overline{Y}for 1≦j≦4. In the exemplar study, each of the 13 VOI ratios for each patient was compared to the controls to assess the degree of overlap in the patient vs. controls there was in the data ranges. Separate weights were calculated for the numerator and denominator VOIs. For example, for the VOIs from the posterior cingulate at [−4, −70, 30], the VOI range for all patients was 0.869-1.895, and the control range was 1.988-2.705. For the patients, 29/32 VOIs were outside the range for the normal group. The VOI with the lowest number falling outside the normal range was at [−24, −12, −28] (left medial temporal), with 17/32. This was set to one by subtracting 16, which was also subtracted from all the weights for other VOIs in the denominator, thus leaving a range for individual weight values, designated as Wᵢ, from 1 for left temporal to 13 for posterior cingulate. A similar process was carried out for weights for VOIs in the numerator, resulting in individual weights, designated Vⱼ, for both components of the ratio, shown in Tables 2 and 3. Once the weights were generated and applied, the weighted VOIs were used to calculate the CDI for the study patients. The weighted VOI ratio was then normalized, such that the grand mean of the VOI ratios from the control group was set to one, and the resulting correction factor was then applied to all weighted VOI ratios. The mean, weighted, normalized VOI ratio constitutes the CDI₁ 400.

$$CDI = {C}_{x} + [ {\sum }_{j = 1}^{n} {V}_{j} {X}_{j} / n ] / [ {\sum }_{i = 1}^{m} {W}_{i} {Y}_{i} / m ]$$

Where, Xⱼ denotes the j^{th} Increased Intensity Value;
Vⱼ denotes the j^{th} Weight for the j^{th} Increased Intensity Value;
Yᵢ denotes the i^{th} Decreased Intensity Value; and
Wᵢ denotes the i^{th} Weight for the i^{th} Decreased Intensity Value.
C_{X} is the correction factor used to normalize the dataset.
   The weights have been established with n=4 and m=9. Once established, this set of weights is used for each new patient presenting for scanning and diagnosis.

The set of steps for calculating CDI₁ are: (1) Import VOI Data Into Spreadsheet 391; (2) Determine Intensity Range Overlap for each VOI Ratio 392; (3) Create Weights for each Intensity Extreme 393; (4) Create Weighted VOI Ratio 394; and (5) Scale and Normalize Ratio 395.

Demographic and screening information are presented in Table 3 for the subject groups used in the referenced exemplar study.

TABLE 3

| | N | F/M | Age (Mean +/− SD) | | Min | Max |
|---|---|---|---|---|---|---|
| Controls | 33 | 10/17 | 51.2 | 17.7 | 19 | 81 |
| Old controls | 19 | 10/9 | 63.9 | 9.3 | 51 | 81 |
| MCI-pros | 5 | 3/2 | 73.0 | 7.7 | 64 | 85 |
| MCI-retro | 12 | 6/11 | 68.2 | 6.4 | 52 | 76 |
| AD | 15 | 6/9 | 66.5 | 9.2 | 53 | 80 |

It was not possible in the study to determine the MMSE scores of all patients identified retrospectively. The group of older controls was used for the SPM analysis, but all controls were included in the CDI results for comparative purposes. For the MCI patients from the pilot study, the mean MMSE was 25.3±2, and the CDT was 3.3±0.8. For the older subset of controls used in the SPM analysis, the mean MMSE was 29.3±0.8, and the mean CDT was 3.8±0.4.

The results of the SPM group analysis of the patients with MCI vs. controls are shown in FIGS. 4 and 5. The retrospective and prospective MCI scan datasets were pooled for this evaluation. Patients were compared to a subset of controls matched for age. FIG. 3 shows the regions of decreased cerebral metabolism that were present in the group analysis. These included many regions characteristic of that seen in previous studies of MCI and AD, including the basal nucleus region, posterior cingulate, bilateral parietal, several left temporal and hippocampus regions, and left frontal regions were found. FIG. 4 shows the regions of increased metabolism in patients with MCI. This includes regions in bilateral motor areas, cerebellum, pons, and a right parietal area that is more medial and superior to the regions found with decreased metabolism. FIGS. 5 and 6 show the maps of significant differences overlaid on canonical brain images, in both cross-sectional (a.) and 3D rendered (b.) views. FIG. 5 shows regions of decreased metabolism, and FIG. 6 shows regions of increased metabolism.

FIG. 8 shows an example of one VOI (posterior cingulate). Data was sampled for VOIs from all 13 regions to calculate the CDI as described above. A comparison of the grouped CDI₁ values was carried out, and the results are shown in Table 4.

TABLE 4

| | Controls | MCI (all) | AD |
|---|---|---|---|
| Mean | 1.000 | 1.112 | 1.224 |
| SD | 0.027 | 0.046 | 0.094 |
| Minimum | 0.949 | 1.051 | 1.076 |
| Maximum | 1.042 | 1.222 | 1.414 |
| Count | 33 | 17 | 15 |
| 95% CL | 0.010 | 0.024 | 0.052 |

As it was unclear whether these data were normally distributed, a Kolmogorov-Smirnov test was carried out and indicated a normal distribution of the data (Table 5):

TABLE 5

| Kolmogorov-Smirnov | Controls | MCI-pros | MCI-retro | AD |
|---|---|---|---|---|
| N | 33 | 5 | 12 | 15 |
| Mean | 1.000 | 1.114 | 1.111 | 1.223 |
| SD | .026 | .048 | .047 | .094 |
| Absolute | .096 | .241 | .161 | .082 |
| Positive | .059 | .236 | .161 | .074 |
| Negative | −.096 | −.241 | −.100 | −.082 |
| KS Z-score | .549 | .540 | .558 | .317 |
| p value (2-tailed) | .924 | .933 | .915 | 1.000 |

Tables 6 (ANOVA) and 7 (t-tests between groups) show statistical analyses for significance:

TABLE 6

| Groups | Count | Sum | Average | Variance |
|---|---|---|---|---|
| Ctrl | 33 | 33 | 1.000 | 0.001 |
| MCI-pros | 5 | 5.570 | 1.114 | 0.002 |
| MCI-retro | 12 | 13.333 | 1.111 | 0.002 |
| AD | 15 | 18.354 | 1.224 | 0.009 |

ANOVA

| Source of Variation | SS | df | MS | F | P-value | F crit |
|---|---|---|---|---|---|---|
| Between Groups | 0.538 | 3 | 0.179 | 60.020 | 3.44E−18 | 2.755 |
| Within Groups | 0.182 | 61 | 0.003 | | | |
| Total | 0.720 | 64 | | | | |

TABLE 7

| Group t-tests | Control | MCI-pros | Control | MCI-retro | Control | AD | MCI-pros | MCI-retro |
|---|---|---|---|---|---|---|---|---|
| Mean | 1.000 | 1.114 | 1.000 | 1.111 | 1.000 | 1.224 | 1.114 | 1.111 |
| Variance | 0.001 | 0.002 | 0.001 | 0.002 | 0.001 | 0.009 | 0.002 | 0.002 |
| Observations | 33 | 5 | 33 | 12 | 33 | 15 | 5 | 12 |
| Df | 36 | | 43 | | 46 | | 15 | |
| T Stat | 7.856 | | 9.901 | | 12.653 | | 0.114 | |
| P(T <= t) two-tail | 2.56E−09 | | 1.17E−12 | | 1.379E−16 | | 0.910563 | |
| T Critical two- | 2.028 | | 2.017 | | 2.013 | | 2.131 | |
| tail | | | | | | | | |

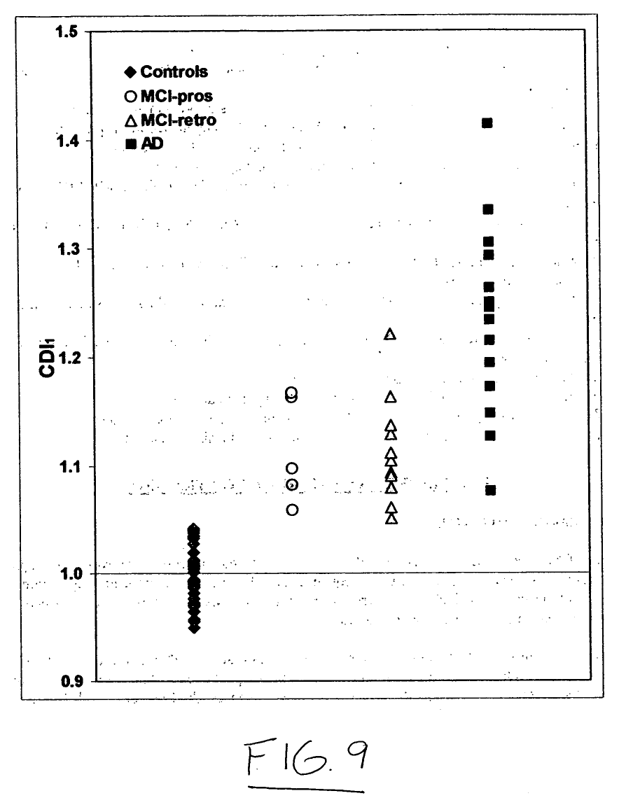

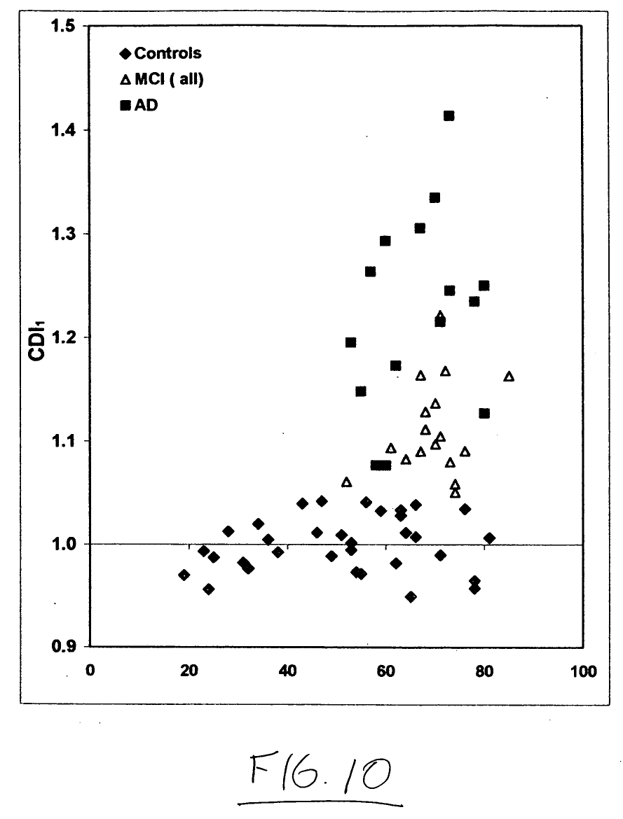

All patient groups compared to controls were highly significant, but there was no difference between patients with early cognitive decline identified retrospectively and those obtained from the pilot MCI study. The normal range for this study was 0.949 to 1.042, (95% CI 0.990-1.010). This critical data range is the embodiment of the normal standard range to which all patient CDI values are compared. The CDI₁ was successful in discriminating 100% of the MCI patients in both the retrospectively and prospectively identified groups (range 1.051-1.222, 95% CI 1.088-1.136), as well as all of probable AD patients (range 1.076-1.414, 95% CI 1.172-1.276). The excellent separation of patients with MCI from controls is shown in FIG. 9. This graph also shows the results for the AD group for comparison. The lack of a relationship of the CDI to age is shown in FIG. 10. There is no correlation with age.

Additional modifications leading to the improvement of the CDI have been investigated using results of the exemplar study. In this effort, the initial weights were used as a baseline for calculation of a second CDI (CDI₂) involving iterative optimization of each weight to maximally separate the study patients from the controls in neural-network fashion. A dynamic table was created where the results of a change of a given weight upon the separation of the groups could be assessed in real-time. Weights were iteratively adjusted with the goal to maximize the separation between the control and MCI populations while minimizing within-group variance. Using this arbitrary method, the weights resulting in optimal separation between the two populations were determined, and are shown in Tables 8 and 9.

TABLE 8

| | Controls | MCI (all) | AD |
|---|---|---|---|
| Mean | 1.000 | 4.595 | 5.659 |
| SD | 0.671 | 1.622 | 3.205 |
| Minimum | −0.687 | 2.976 | 2.315 |
| Maximum | 2.237 | 7.376 | 13.252 |
| Count | 33 | 17 | 15 |
| 95% CI | 0.238 | 0.834 | 1.775 |

TABLE 9

| Group t-tests | Controls | MCI-pros | Controls | MCI-retro | Controls | AD | MCI-pros | MCI-retro |
|---|---|---|---|---|---|---|---|---|
| Mean | 1.000 | 5.010 | 1.000 | 4.432 | 1.000 | 5.659 | 5.010 | 4.432 |
| Variance | 0.451 | 3.933 | 0.451 | 2.255 | 0.451 | 10.271 | 3.933 | 2.255 |
| Observations | 33 | 5 | 33 | 12 | 33 | 15 | 5 | 12 |
| df | 36 | | 43 | | 46 | | 15 | |
| t Stat | −9.131 | | −10.662 | | −8.068 | | 0.660 | |
| P(T <= t) one-tail | 3.33E−11 | | 5.95E−14 | | 1.17E−10 | | 0.260 | |
| t Critical one-tail | 1.688 | | 1.681 | | 1.679 | | 1.753 | |
| P(T <= t) two-tail | 6.66E−11 | | 1.19E−13 | | 2.35E−10 | | 0.519 | |
| t Critical two-tail | 2.028 | | 2.017 | | 2.013 | | 2.131 | |

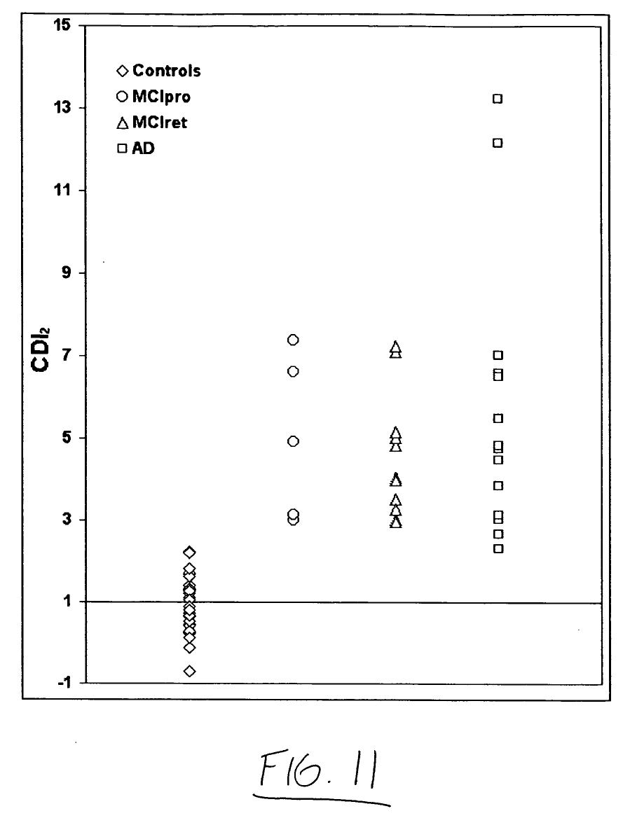

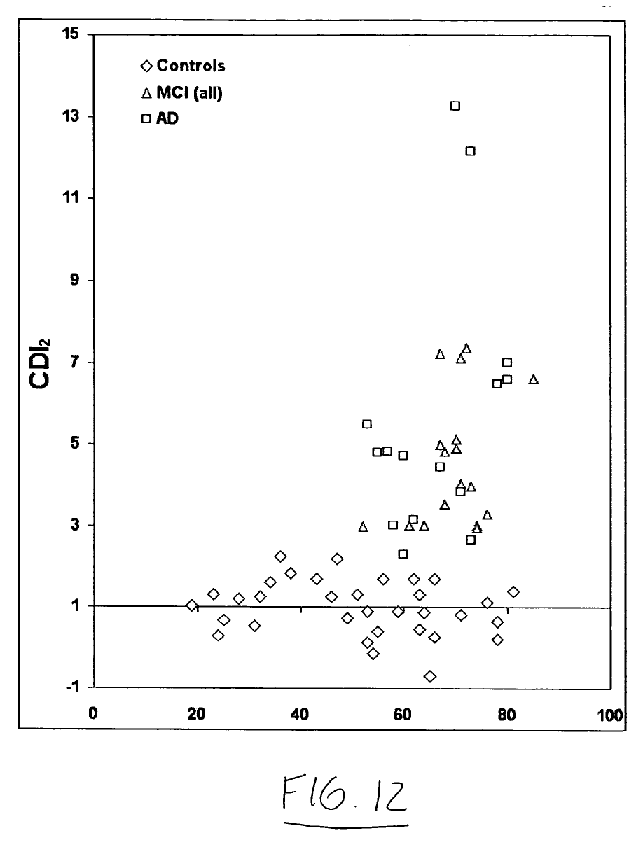

Results from the group analysis using CDI₂are shown in Table 8, and are presented graphically in FIGS. 11 and 12. Statistical significance is presented in Table 9. While this method of CDI creation does result in the best discrimination of MCI patients from older controls, it did not discriminate patients with AD as well as CDI₁.

The SPM analysis as discussed above is valuable, but has some significant drawbacks. Determination of significance is somewhat arbitrary. The most conservative significance level is non-a-priori, based upon the intensity at the single-voxel level, and typically requires an SPM(Z) statistic in the 4.5 to 5 range to be determined truly significant. The least conservative significance level is based on an a priori hypothesis about activity in a given region, examines spatial extent or a combination of extent and peak height more so than intensity, and can be as low as a Z score of perhaps 2.5. It was noted that a characteristic pattern of decreased metabolism could emerge if the significance level was set to low levels. This “trend” in characteristic patterns was what was seen in several patients with MCI, where there were no obvious clinically defined lesions. Because of the problematic statistical significance question involved, and because it can be difficult to interpret a pattern of activity, especially in the light of low thresholds, it was necessary to create a way of objective examination of the PET brain image data that is more definitive and usable on the single patient level. Because analysis of ROIs removes the major problem of multiple comparisons and conservative Bonferroni adjustments, this method was the main focus of further research beyond SPM and led to the methodology discussed in the preferred embodiment.

Using ROIs to examine both semi-quantitative and absolute brain image data was once the major methodology in use (and is still quite common), as the voxel-by-voxel approach incorporated in SPM is a relatively novel method. Most studies of semi-quantitative image data intensity normalized the data by dividing a given ROI value by that obtained from the pons (e.g., de Santi et al, 2001), the cerebellum (e.g., Cappa et al, 2001), some other “standard” (presumptively unaffected) region, such as the sensorimotor cortex (e.g., Arnaiz et al, 2001), or by an estimate of the global value (de Leon et al, 2001). It is interesting to note that in the research presented here, the cerebellum, pons, and sensorimotor area were all found to have increased activity in the SPM analysis when patients with MCI were compared to control subjects. All these regions, as outlined above, have been used as reference regions in studies of AD, in the belief that they are preserved and thus represent normal rates of metabolism. Alternatively, it is possible to look at the relationship between two regions on opposite sides of the brain by creating a ratio, as in the “asymmetry index” (Russel et al, 1997). One of the major problems of ROI analysis of functional imaging data (pre-SPM) is that definition of the shape, size, and location of the ROI is often subjective and arbitrary. There are many variations on this theme extant in the literature. In essence, using ROIs arbitrarily predefine a hypothetical lesion. For example, if an ROI 2 cm in size is arbitrarily placed in the temporal lobe to interrogate for a region of hypometabolism, and is positioned over a portion of a 1 cm lesion, then the (averaged) intensity value from the ROI will have increased variance due to being the mean of voxels from outside the lesion that are averaged together with voxels from within the lesion. The end result may be an ROI value that lacks significance. The solution to this problem is to place ROIs in positions where there is known pathology, e.g., based on the results of an SPM analysis. ROIs have been previously derived from SPM regional maxima (Buchel and Friston, 1997). Thus, the use of ROIs in the instant invention, has evolved past the earlier usage. The employment of ROIs in the instant invention escapes the major problem inherent in previous of SPM analyses (multiple comparisons), and also escapes the main problem historically associated with ROI analysis of having an arbitrary location in relation to the suspected pathology.

The CDI was derived by examining the regions found to be significant, or trending towards significance in the SPM analysis. All regions used for the CDI were derived from the SPM analysis. While it may be possible to obtain an equally valid CDI with more or fewer regions, arriving at the 9 regions of decreased metabolism and 4 regions of increased metabolism was essentially arbitrary. These were the major regions that separated out of the SPM analysis of older controls vs. MCI patients. The number of regions used was derived selecting SPM-defined regions of maximal difference, and determining which of these regions had the highest degree of separation between the two groups (frequency analysis).

The CDI of the instant invention is unique because it is constructed using weights based upon the frequency of intensity abnormalities found in the 13 regions. Whereas most ratios are between one region under examination and another region used as a standard, the ratio in the CDI is derived from the mean of four of the weighted ROIs divided by the mean of the other nine weighted ROIs. All ROIs are being examined experimentally; there are none that are arbitrarily chosen as the “standard” region or regions. This use of ROIs has not been taught previously in the art. Thus, forming a mean for the numerator and denominator is novel, derivation of the weights is novel, and using ROIs from increases for the numerator and ROIs from decreases for the denominator is novel. Forming a mean, weighted, normalized ratio is thus a unique approach in the detection of MCI. While several of the regions derived from the SPM analysis are consistent with those reported in the literature as being involved in MCI/AD pathophysiology (e.g., NBM, medial temporal, posterior cingulate, superior parietal) several of them, especially regions of increased activity, have not previously been reported, and are thus essentially novel to this method. Moreover, while there have been anecdotal reports of sensorimotor cortex preservation in AD (Arnaiz et al, 2001), no one has previously reported increased activity in this region from an SPM analysis, related to MCI.

It has become apparent from many studies appearing in the functional imaging literature that the cerebellum often plays a major role in cognitive as well as it's more well known motor functions (Parsons et al, 1997; Rapoport et al, 2000). The neo-cerebellum has undergone striking parallel evolution with the neo-cortex, particularly in combination with the major frontal lobe expansion unique to humans. While the exact function of this expanded cerebellum remains to be established, the sheer size and magnitude of the corticopontocerebellar connections give a clue to its likely involvement in cognitive processes (Leiner et al 1986, 1989). It has been implicated in language processing (Leiner et al, 1991) and there is anatomical evidence supporting a role for the cerebellum in cognition (Middleton and Strick 1994; Schmahmann and Pandya, 1995). Many functional neuroimaging studies demonstrate cerebellar involvement in cognitive processes. In cognitive activation studies that include the cerebellum, it is common to find increased activity in the cerebellum. One of the pioneer studies on the role of cerebellum in cognition indicated involvement of certain cerebellar regions in processing of sensory information rather than fine motor control (Gao et al, 1996). The cerebellum receives cortical afferents via the pontine relay nuclei. These afferents have recently been discovered to come from more widespread areas of the cortex than was originally thought, and seem to be reciprocal (Schmahmann and Pandya, 1997). Thus, a network exists to support involvement in cognition. Cerebellar lesions have been linked to a cognitive affective syndrome (Schmahmann and Sherman, 1998). This report of twenty patients with disease confined to the cerebellum found striking cognitive and behavioral deficits including difficulties with verbal fluency, working memory, visuospatial organization, personality changes and blunting of affect, in addition to other changes. Cerebellar changes have also been found before in dementia. A recent PET study found decreased cerebellar metabolism in patients with severe Alzheimer's, however they also found significant declines in glucose metabolism throughout the cerebrum (Ishii et al, 1997). The magnitude of the metabolic changes seen was least in the cerebellum, and greatest in the parietal cortex. The cerebellar changes found were only significant in patients with severe Alzheimer's. Another recent examination of patients with olivopontocerebellar atrophy found that this group had deficits in tasks requiring intact frontal and parietal cortices. They postulated that the cerebellum was involved in modulation of these cortical areas, and thus the atrophy had resulted in the cognitive changes seen (Arroyo-Anllo and Botez-Marquard, 1998). A case report of a patient with a cyclic cognitive-affective syndrome examined cerebral perfusion using SPECT (Patterson, 2001). This patient with atypical symptoms of dementia shows increased flow in the cerebellum, which may represent increased activity of the Purkinje cell's inhibitory output, or increased activity of cells upstream to the Purkinje neurons. The increase may have been compensatory, secondary to deficits in other interconnected areas such as the posterior parietal lobe.

In the exemplar study, relative increases in metabolism are reported in the pons, cerebellum, and motor area of patients with MCI. There are previously reported findings of increased cerebellar metabolism in patients with AD (Patterson et al, 2002). There have been numerous works that report the use of the cerebellum as a reference or control region for normalizing semi-quantitative PET or SPECT data. Indeed, there has been debate on this topic, and at least one previous study has been done to validate the use of the cerebellum as a reference region in AD (Pickut et al, 1999). Others have found either no change (Pickut et al 1999, Soonawala et al, 2002), or decreased cerebellar metabolism in AD (Ishii et al, 1997). One further study found that pontine metabolism was most preserved in patients with AD compared to controls (Minoshima et al, 1995b). The use of a ratio of cerebellar to brain activity is not a novel methodology, in fact it is a standard means of “normalizing” semi-quantitative data (see above section on ROIs). As our work here is semi-quantitative, it is possible that the increases found in the pons, cerebellum, and motor strip are the result of global declines in the MCI population, sparing these regions. It is also possible that the metabolic decline found in some regions (posterior cingulate, parietal, etc) have resulted in compensatory activity in other nodes in a network of brain regions. There have been numerous reports in the literature documenting the involvement of the cerebellum in cognition (see Rapoport et al, 2000 for review), so it is not necessarily safe to presume that the cerebellum is uninvolved in AD or other disorders involving cognition. We postulate here that our results may not simply be intensity normalization due to global changes, but compensatory increased activity. Further study on this question using absolute glucose metabolic rate and/or structural equation modeling to examine nodal interactions is certainly warranted, but whether or not the actual metabolism in these regions is increased or normal, they still serve as optimal regions for the calculation of the CDI.

ROI data based on SPM results has been used to examine functional connectivity between the cerebellum and other regions in a study of acute psychosis and response to antipsychotic medication. Functional connectivity analysis is simply looking at the correlation coefficient between two regions (e.g., the cerebellum and the left dorsolateral prefrontal cortex) in a population, and comparing that value to one obtained in another population. This data has not been published as it contains some admittedly serious confounds. However, the underlying method of using SPM maxima to define ROIs, and then using the ROI data to look at the relationship between two or more brain regions is still valid. The concept of using a region of activity as a locus for an ROI is extant in the literature, and has been used previously (Buchel et al, 1997). This study used the locations of regional maxima from an SPM analysis as seed points for ROIs, and this data was then entered into a Structural Equation Model (SEM) analysis. The SEM and similar methodologies are more advanced than simple functional connectivity-type correlation analyses, and are called “effective connectivity” analyses. This method is important to review as it bears some similarities to my method. SEM involves the use of a set of mean intensity values derived from ROIs typically taken from specific regions. These regions can be defined by SPM maxima, as in the cited study. The underlying concept is based upon defined anatomical connections, and thus interprets a relationship between two regions as composed of either a direct connection, an indirect connection, or (more commonly) a combination of the two. This is important as the addition of greater than three “nodes” in this network increases the complexity of the calculation by a least an order of magnitude, and this complexity increases in a geometric fashion for each node added. One similarity to my method is that weights are assigned to a given “path” between two nodes. These weights are used to calculate a path coefficient that represents the strength or activity of the connection between the two nodes. This type of analysis is useful to examine the relationship between several regions, and how it may change with different cognitive activities. The CDI samples mean ROI intensity data from multiple regions, and those values are entered into a formula to calculate the CDI. While one component of the formula involves weights, this is not to examine the relationship between regions. While there may be a relationship between certain regions sampled for the CDI, this is not implied, intrinsic, or necessary for the CDI to be valid and functional.

As discussed before, three previous reports indicate that it is possible to detect brain metabolic changes using PET across groups of premorbid patients, who have not yet developed MCI, before subjective symptoms or neuropsychological impairment occurs (De Leon et al, 2001; Reiman et al, 2001; Small et al, 2000). All three use group analysis to detect changes in groups of subjects. The SPM results used in the instant invention are highly consistent with these previous reports. One study examined brain scans in a post-hoc measures, separating them based upon whether they develop AD later in life. The other two were also longitudinal studies. None of the three present a methodology that enables evaluation and production of a measure that is usable in a single patient. De Leon and others followed 48 healthy elderly individuals, and scanned them at baseline and again after 3 years. Some of the subjects showed evidence of cognitive decline. By grouping these patients post-hoc, and looking at their first scans as a function of whether they developed cognitive decline at 3 years, they found decreased metabolism in the entorhinal cortex, as well as increased frequency of ApoE4+ genotype. The Reiman study followed normal subjects who either had or didn't have the ApoE4 phenotype, and scanned twice with a two-year interval. They found that subjects who were ApoE4+ had decreased metabolism in regions of the temporal lobe, posterior cingulate, prefrontal cortex, basal forebrain, parahippocampus, and thalamus, in regions similar to those found in the present study. The last study by Small and others was similar. They followed 61 subjects, 54 of whom were aware of mild memory loss, but who were “normal” as determined by cognitive tests. In this population, ApoE4+ genotype was associated with initial decreased metabolism in the posterior cingulate, inferior parietal and lateral temporal areas. These metabolic changes predicted cognitive decline. These studies show that PET scanning using FDG is the most sensitive measure known for the detection of this disorder. Previous data indicates that using PET to diagnose early AD was cost-effective and no more expensive than other methods, and resulted in improved accuracy (Silverman et al, 2002). If our data holds up in further study, then PET will become even more accurate in the diagnosis of MCI.

There have been previous reports that have attempted to discriminate patients with early cognitive changes or AD from normal subjects by using various methods of objective analysis. A study using a diagnostic index based on parietal lobe Z-scores was able to detect 97% of AD patients (Minoshima et al, 1995a). This same group extended this technique to patients with isolated memory impairment, but were able to detect only 50% (Berent et al 1999). Another report that used multiple regression and discriminant analysis correctly identified 87% of patients with mild to moderate AD and controls (Azari et al, 1993). Another study that used logistic regression identified 95% of AD patients using a combined regressor of FDG metabolic data (from an arbitrarily defined ROI in the left temporoparietal area), along with performance on a “block design” cognitive test (Arnaiz et al 2001). A SPECT study using singular value decomposition and discriminant function analyses was able to detect about 60% of patients with early AD/MCI (Johnson et al, 1998).

The findings from all of these studies can be distinguished from the findings of the exemplar study presented here and the methodology of the instant invention by the variability in mathematical approaches, sensitivity, and most important and unique in the instant invention of the use of SPM-derived regional maxima as loci for the VOIs. This method eliminates the confounding condition of arbitrarily defining the lesion site, as well as bypasses the confounding requisite Bonferroni correction for multiple comparisons in SPM. Our method of using a CDI based on multiple VOIs allows for variance across the presentation, while methods that examine only one region (e.g., the parietal lobe) do not. The method of the preferred embodiment of the instant invention also examines 13 major nodes that we believe are most affected by the processes of AD, based on the a priori knowledge gleaned from the SPM analysis.

The data presented here for the CDI₂made use of an iterative optimization technique for VOI weighting that bears some similarity to neural-network classification. There have been two previous studies that used a neural-network method (Kippenham, et al, 1992, 1994) to classify patients with AD from controls. These two reports used neural-network models based on 67 ROIs drawn in all the major regions of the brain. The 1992 study reported that, for patients with possible AD (MMSE of 19±8), the area under the relative operating curve (ROC) was 0.81, similar to that for the clinical evaluation. This was somewhat higher for probable AD (MMSE 15±7) with a ROC area of 0.85, and improved even more in the 1994 study by using a scanner with higher resolution (ROC area 0.95). One important difference between the Kippenham studies and the data presented here is that we used a priori knowledge of where the pathological regions were (determined with SPM) to sample VOI data. In the instant invention, the SPM methodology is utilized in a unique way, to determine the exart location of regionally significant change for a 3D spherical VOI. By doing this, it is possible to bypass the major drawback of using ROI analysis, which is that the ROI is typically drawn arbitrarily. Even when drawn based upon anatomically defined areas, there still is no assurance that an area so defined will respond homogenously and thus provide a homogenous response. The use of VOIs in the instant invention is determined in this manner, along with the weights used to calculate the CDI are likely responsible for the very high sensitivity present in our data.

PET is a technology that can make use of a variety of radioligands, and is not limited to FDG. AD has been studied with radioligands that bind to cholinergic receptors (e.g., Shinotoh et al, 2003) as well as to neurofibrillary plaques (Shoghi-Jadid et al, 2002). These techniques are quite different than the results presented here as they make use of completely different radioligands and do not examine brain metabolism.

The CDI as described in the preferred embodiment is a marker that can be used to predict AD. As stated above, the marker/method described in U.S. Pat. Nos. 5,873,823 and 5,632,276 may be applied to the detection and diagnoses of multiple disease states, including both PD and AD. To apply the teachings of the instant patent to identify and predict the onset of more severe symptoms in other conditions such as PD, change would be made to the methodology of the instant invention to use different VOI's. This is due to the fact that the SPM analysis in the instant patent provides loci that are specific for MCI, and thus the loci sampled as used in the CDI are specific for MCI and AD. For diagnoses of PD, an SPM analysis of patients with PD compared to control would have to be completed.

With this requirement established, the following examples illustrate extensions of the CDI to additional clinical presentations.

Parkinson's Disease (PD)

PD is a disorder of the brain which affects the dopaminergic neurons of the brainstem first and foremost. By the time that patients first notice a movement disorder or feel the first clinically noticeable signs and symptoms, 50% of the dopamine neurons have been destroyed. Previous reports have shown that there are metabolic changes present in the cerebral cortex as well as subcortical structures in early PD. One specific report by Eidelberg, as reported above, and others used FDG-PET and Scaled Subprofile Modeling to make predictions about disease states in PD. The description of the methodology in both the Eidelberg patent and the manuscript lacks clarity.

The methodology of the instant patent may be applied to gather data in the same fashion with early PD as was done with patients with MCI. Baseline FDG PET scans can be obtained for a group of patients presenting with these symptoms or complaints, and compared to a group of age matched controls using SPM. The SPM statistical data can be used to determine the location (specific coordinates) of regions of significant change. These regions can be sampled with a 5 mm radius VOI using MARSBAR™. Estimates of the frequency of abnormality of each region can be calculated across the patient sample, and used to generate weights for each region. The mean of the weighted VOIs from regions where significant increased metabolism is found can be divided by that from regions with decreased metabolism. The grand mean of this ratio in the control subjects can be adjusted to 1, and the resulting adjustment factor used to normalize all ratios to this standard. This value can be called the “Parkinson's Disease Index,” or PDI.

Closed-Head Injury (CHI)

CHI is an altogether too common affliction. Patients who have suffered from concussive illnesses often have very little if any objective evidence on an MRI or CT scan to indicate that a traumatic injury has occurred. However, neuropsychological tests and behavioral measures often do find sometimes subtle changes. The purpose of using a PET index in CHI is to provide a definitive and objective measure that can guide treatment and prognosis.

The CDI methodology may be further adapted for use in the objective analysis of functional brain data (FDG-PET scans) from patients with CHI. This would involve the use of VOIs from regions that vary on a per-patient basis, as potential metabolic lesions would vary from patient to patient, depending on the location of the traumatic insult and the degree of coup/contre-coup type injury. Baseline FDG PET scans can be obtained for a given patient, and each individual patient can be compared to a group of controls using SPM. The SPM statistical data can be used to determine the location (specific coordinates) of regions of significant change. These regions can be sampled with a 5 mm radius VOI using MARSBAR™. The mean of the VOIs from regions where significant increased metabolism was found can be divided by that from regions with decreased metabolism. The grand mean of this ratio in the control subjects can be adjusted to 1, and the resulting adjustment factor used to normalize all ratios to this standard.

Substance Abuse

Abuse of dangerous and illicit substances is often found to be associated with pathophysiology in the orbitofrontal cortex and associated limbic and paralimbic regions. While the specific regions may vary with the substance being abused, the rationale remains the same. The purpose of using a PET index in patients who have abused drugs is potentially manifold: to investigate the risk of addiction in certain populations, to study the effect that acute substance abuse or dependency has on cerebral metabolism, and to evaluate populations for lesions who are abstinent but who have abused or were dependent in the past.

Baseline FDG PET scans would be obtained for a group of patients with a history of drug use, and compared to a group of age matched controls using SPM. The SPM statistical data can be used to determine the location (specific coordinates) of regions of significant change. These regions can be sampled with a 5 mm radius VOI using MARSBAR™. Estimates of the frequency of abnormality of each region can be calculated across the patient sample, and used to generate weights for each region. The mean of the weighted VOIs from regions where significant increased metabolism is found can be divided by that from regions with decreased metabolism. The grand mean of this ratio in the control subjects can be adjusted to 1, and the resulting adjustment factor used to normalize all ratios to this standard.

Lewy Body Dementia, Pick's Dementia, Huntington's Disease Three other less common dementing diseases are Lewy Body Dementia, Pick's Dementia, and Huntington's Disease. These diseases have characteristic metabolic lesion patterns on the PET scan, and thus it is quite feasible to propose Cognitive Decline Indices that are specific for the exact dementia type. The purpose of using a specific PET index for these types of dementia would be several fold: to detect the dementing process as early as possible, to discriminate which type of dementing process it is, and to facilitate the early treatment of these disease processes.

Baseline FDG PET scans can be obtained for a group of patients presenting with these symptoms or complaints, and compared to a group of age matched controls using SPM. The SPM statistical data can be used to determine the location (specific coordinates) of regions of significant change. These regions can be sampled with a 5 mm radius VOI using MARSBAR™. Estimates of the frequency of abnormality of each region can be calculated across the patient sample, and used to generate weights for each region. The mean of the weighted VOIs from regions where significant increased metabolism was found can be divided by that from regions with decreased metabolism. The grand mean of this ratio in the control subjects can be adjusted to 1, and the resulting adjustment factor used to normalize all ratios to this standard.

In developing the CDI for any of these conditions, a patient's CDI is compared to established normal ranges of values. The presence of normality or abnormality is determined from the CDI value 500. If the CDI reading is negative, the patient is advised and educated about the clinical course of potential illnesses and told the signs to watch for. The potential benefits of preventative measures including anti-oxidants, mental exercises, beneficial diet and adequate rest are discussed 510. If the CDI reading is positive, the patient is educated about the meaning of the positive reading, and informed about the projected clinical course of the illness. The benefits of medication, and the potential benefit of ameliorative measures such as anti-oxidants, mental exercises, beneficial diet and adequate rest are discussed. 520. In either case, results are given to the referring physician and the patient is scheduled for re-evaluation 530. Patient data is stored in the comprehensive patient database 540.

The above descriptions of the exemplary embodiments of methods for the determination of clinical conditions and for the quantitative description of metabolically correlated brain function are for illustrative purposes. Those skilled in the art who have the benefit of this disclosure will recognize that certain changes can be made to the component parts of the method of the present invention without changing the manner in which those parts function to achieve their intended result. The instant invention may also be practiced in the absence of any element not specifically disclosed. All such changes, and others which will be clear to those skilled in the art from this description of the preferred embodiments of the invention, are intended to fall within the scope of the following, non-limiting claims.

### References

Arnaiz E, Jelic V, Almkvist O, Wahlund L, Winblad B, Valind S, and Nordberg A (2001). Impaired cerebral glucose metabolism and cognitive functioning predict deterioration in mild cognitive impairment. Neuroreport, 12(4):851-855.

Arroyo-Anllo E M and Botez-Marquard T B: Neurobehavioral Dimensions of Olivopontocerebellar Atrophy. J Clin Exp Neuropsychol. 1998; 20:52-59.

Azari N P, Pettigrew K D, Schapiro M B, Haxby J V, Grady C L, Pietrini P, Salerno J A, Heston L L, Rapoport S I, Horwitz B. Early detection of Alzheimer's disease: a statistical approach using positron emission tomographic data. J Cereb Blood Flow Metab 1993 May;13(3):438-47

Berent S, Giordani B, Foster N, Minoshima S, Lajiness-O'Neill R, Koeppe R, Kuhl D E. Neuropsychological function and cerebral glucose utilization in isolated memory impairment and Alzheimer's disease. J Psychiatr Res 1999 January-February;33(1):7-16

Boellaard R, van Lingen A, and Lammertsma A (2001). Experimental and Clinical Evaluation of Iterative Reconstruction (OSEM) in Dynamic PET: Quantitative Characteristics and Effects on Kinetic Modeling. J Nucl Med, 42:808-817.

Brett M, Anton J L, Valabregue R, Poline J B (2002). Region of interest analysis using an SPM toolbox [abstract] Presented at the 8th International Conferance on Functional Mapping of the Human Brain, Jun. 2-6, 2002, Sendai, Japan. Neurolmage, Vol 16, No 2.

Brookmeyer R, Gray S, Kawas C. (1998). Projections of Alzheimer's disease in the United States and the public health impact of delaying disease onset. Am J Public Health. 88(9):1337-42.

Buchel C, & Friston K J, (1997). Modulation of connectivity in visual pathways by attention: cortical interactions evaluated with structural equation modelling and fMRI. Cerebral Cortex, 7, 768-778.

Chen P, Ratcliff G, Belle S H, Cauley J A, DeKosky S T, Ganguli M, (2001). Patterns of Cognitive Decline in Presymptomatic Alzheimer Disease: A Prospective Community Study. Arch Gen Psych, 58:853-858.

Collie A, Maruff P. An analysis of systems of classifying mild cognitive impairment in older people. Aust N Z J Psychiatry 2002 February;36(1):133-40

De Leon M J, Convit A, Wolf O T, Tarshish C Y, DeSanti S, Rusinek H, Tsui W, Kandil E, Scherer A J, Roche A, Imossi A, Thorn E, Bobinski M, Caraos C, Lesbre P, Schlyer D, Poirier J, Reisberg B, Fowler J. (2001). Prediction of cognitive decline in normal elderly subjects with 2-[(18)F]fluoro-2-deoxy-D-glucose/poitron-errission tomography (FDG/PET). Proc Natl Acad Sci U S A. 98(19):10966-71.

De Santi S, de Leon M J, Rusinek H, Convit A, Tarshish C Y, Roche A, Tsui W H, Kandil E, Boppana M, Daisley K, Wang G J, Schlyer D, Fowler J. (2001). Hippocampal formation glucose metabolism and volume losses in MCI and AD. Neurobiol Aging, 22(4):529-539.

Eidelberg D, Moeller J R, Ishikawa T, Dhawan V, Spetsieris P, Chaly T, Belakhlef A, Mandel F, Przedborski S, Fahn S. (1995a). Early differential diagnosis of Parkinson's disease with 18F-fluorodeoxyglucose and positron emission tomography. Neurology, 45(11):1995-2004.

Eidelberg D, Moeller J R, Ishikawa T, Dhawan V, Spetsieris P, Chaly T, Robeson W, Dahl J R, Margouleff D. (1995b). Assessment of disease severity in parkinsonism with fluorine-18-fluorodeoxyglucose and PET. J Nucl Med. 36(3):378-83.

Evans D A, Funkenstein H H, Albert M S, Scherr P A, Cook N R, Chown M J, Hebert L E, Hennekens C H, Taylor J O. (1989). Prevalence of Alzheimer's disease in a community population of older persons. Higher than previously reported. JAMA, 262(18):2551-6.

Folstein M F, Folstein, S E and McHugh P R (1975) Mini-Mental State: A practical method for grading the state of patients for the clinician, Journal of Psychiatric Research, 12: 189-198.

Fox P T, Raichle M E, Mintun M A, Dence C (1988). Nonoxidative glucose consumption during focal physiologic neural activity. Science 241:462-464.

Friston K J, Ashburner J, Frith C D, Poline J B, Heather J D, and Frackowiak R S J (1995a). Spatial registration and normalization of images. Human Brain Mapping, 2:165-189.

Friston K J, Holmes A P, Worsley K J, Poline J B, Frith C D, and Frackowiak R S J (1995b). Statistical parametric maps in functional imaging: A general linear approach. Human Brain Mapping, 2:189-210.

Gao J H, Parsons L M, Bower J M, Xiong J, Li J, Fox P T (1996). Cerebellum implicated in sensory acquisition and discrimination rather than motor control. Science, 272(5261):545-7.

Ibanez V, Pietrini P, Alexander G E, Furey M L, Teichberg D, Rajapakse J C, Rapoport S I, Schapiro M B, Horwitz B. Regional glucose metabolic abnormalities are not the result of atrophy in Alzheimer's disease. Neurology 1998June;50(6):1585-93

Ishii K, Sasaki M, Kitagaki H, Yamaji S, Sakamoto S, Matsuda K, Mori E. Reduction of cerebellar glucose metabolism in advanced Alzheimer's disease. J Nucl Med. 1997June;38(6):925-8.

Johnson K A, Jones K J, Becker J A, Satlin A, Holman B L, Albert M S (1998). Preclinical prediction of Alzheimer's disease using SPECT. Neurology, 50:1563-1571.

Katzman R, Kang D, Thomas R. (1998). Interaction of apolipoprotein E epsilon 4 with other genetic and non-genetic risk factors in late onset Alzheimer disease: problems facing the investigator. Neurochem Res. 1998March;23(3):369-76.

Kippenhan J S, Barker W W, Nagel J, Grady C, Duara R. Neural-network classification of normal and Alzheimer's disease subjects using high-resolution and low-resolution PET cameras. J Nucl Med. 1994January;35(1):7-15.

Kippenhan J S, Barker W W, Pascal S, Nagel J, Duara R. Evaluation of a neural-network classifier for PET scans of normal and Alzheimer's disease subjects.J Nucl Med. 1992 August;33(8):1459-67.

Kirby M, Denihan A, Bisrce I, Coakley D, Lawlor B A. The clock drawing test in primary care: sensitivity in dementia detection and specificity against normal and depressed elderly. Int J Geriatr Psychiatry. 2001October;16(10):935-40.

Leiner H C, Leiner A L, Dow R S (1986): Does the cerebellum contribute to mental skills? Behav Neurosci, 100(4):443-54.

Leiner H C, Leiner A L, Dow R S (1989): Reappraising the cerebellum: what does the hindbrain contribute to the forebrain? Behav Neurosci, 103(5):998-1008.

Leiner H C, Leiner A L, Dow R S (1991): The human cerebro-cerebellar system: its computing, cognitive, and language skills. Behav Brain Res, 44(2):113-28.

Meltzer C C, Zubieta J K, Brandt J, Tune L E, Mayberg H S, Frost J J. Regional hypometabolism in Alzheimer's disease as measured by positron emission tomography after correction for effects of partial volume averaging. Neurology. 1996August;47(2):454-61.

Middleton F A and Strick P L (1994): Anatomical evidence for cerebellar and basal ganglia involvement in higher cognitive function. Science, 266(5184):458-461.

Minoshima S, Frey K A, Koeppe R A, Foster N L, Kuhl D E. A diagnostic approach in Alzheimer's disease using three-dimensional stereotactic surface projections of fluorine-18-FDG PET. J Nucl Med. 1995July,36(7):1238-48.

Minoshima S, Frey K A, Foster N L, Kuhl D E. Preserved pontine glucose metabolism in Alzheimer disease: a reference region for functional brain image (PET) analysis. J Comput Assist Tomogr. 1995b July-August;19(4):541

Nadeau S E, Crosson B (1995). A guide to the functional imaging of cognitive processes. Neuropsychiatry, Neuropsychology and Behavioral Neurology 8:143-162.

Parsons L M and Fox P T: Sensory and cognitive functions. Int Rev Neurobiol 1997; 41:255-271.

Patterson J C (2001): Cerebellar perfusion abnormalities correlated with change in cognitive and affective state in a 78-year-old man. Am J Geriatr Psychiatry. 2001Summer;9(3):309-14.

Patterson J C, Tainter K H, Lilien D L, and Glabus M F. (2002). Increased Cerebellar Metabolism in Alzheimer's disease. (abstact). Presented at the 8th International Conference on Functional Mapping of the Human Brain, Jun. 2-6, 2002, Sendai, Japan. Available on CD-Rom in NeuroImage, Vol. 16, No. 2.

Petersen R C, Stevens J C, Ganguli M, Tangalos E G, Cummings J L, DeKosky S T. (2001). Practice parameter: early detection of dementia: mild cognitive impairment (an evidence-based review). Report of the Quality Standards Subcommittee of the American Academy of Neurology. Neurology. 56(9):1133-1142.

Pickut B A, Dierckx R A, Dnbbeleir A, Audenaert K, Van Laere K, Vervaet A, De Deyn P P. Validation of the cerebellum as a reference region for SPECT quantification in patients suffering from dementia of the Alzheimer type. Psychiatry Res. 1999 April 26;90(2):103-12.

Rapoport M, van Reek-um R, Mayberg H (2000). The Role of the Cerebellum in Cognition and Behavior: A Selective Review. J Neuropsych Clin Neurosci 12:193-198.

Reiman E M, Caselli R J, Chen K, Alexander G E, Bandy D, Frost J. (2001). Declining brain activity in cognitively normal apolipoprotein E epsilon 4 heterozygotes: A foundation for using positron emission tomography to efficiently test treatments to prevent Alzheimer's disease. Proc Natl Acad Sci U S A. 98(6):3334-9.

Russell J M, Early T S, Patterson J C, Martin J L, Villanueva-Meyer J, McGee M D, (1997). Temporal lobe perfusion asymmetries in schizophrenia. J Nucl Med, 38(4):607-12.

Schmahmann J D and Pandya D N (1995). Prefrontal cortex projections to the basilar pons in rhesus monkey: implications for the cerebellar contribution to higher function. Neurosci Lett, 199(3):175-8.

Schmahmann J D and Pandya D N: The cerebrocerebellar system. Int Rev Neurobiol. 1997; 41:31-60.

Schmahmann J D and Sherman J C: The cerebellar cognitive affective syndrome. Brain 1998; 121:561-579.

Schwartz W J, Smith C B, Davidsen L, Savaki H, Sokoloff L, Mata M, Fink D J, Gainer H (1979). Metabolic mapping of functional activity in the hypothalamo-neurohypophysial system of the rat. Science 205:723-725.

Shinotoh H, Fukushi K, Nagatsuka S, Tanaka N, Aotsuka A, Ota T, Namba H, Tanada S, Irie T (2003). The amygdala and Alzheimer's disease: positron emission tomographic study of the cholinergic system.Ann N Y Acad Sci, 985:411-419.

Shoghi-Jadid K, Small G W, Agdeppa E D, Kepe V, Ercoli L M, Siddarth P, Read S, Satyamurthy N, Petric A, Huang S C, Barrio JR (2002). Localization of neurofibrillary tangles and beta-amyloid plaques in the brains of living patients with Alzheimer disease. Am J Geriatr Psychiatry 10(1):24-35.

Shulman K, Shedletsky R, Silver I. The challenge of time: clock-drawing and cognitive function in the elderly. Int J Geriatr Psychiatry 1986;1:135-40.

Silverman D H, Gambhir S S, Huang H W, Schwimmer J, Kim S, Small G W, Chodosh J, Czernin J, Phelps M E. Evaluating early dementia with and without assessment of regional cerebral metabolism by PET: a comparison of predicted costs and benefits. J Nucl Med. 2002 February;43(2):253-66.

Small G W, Ercoli L M, Silverman D H, Huang S C, Komo S, Bookheimer S Y, Lavretsky H, Miller K, Siddarth P, Rasgon N L, Mazziotta J C, Saxena S, Wu H M, Mega M S, Cummings J L, Saunders A M, Pericak-Vance M A, Roses A D, Barrio J R, Phelps M E. (2000). Cerebral metabolic and cognitive decline in persons at genetic risk for Alzheimer's disease. Proc Natl Acad Sci U S A. 97(11):6037-42.

Soonawala D, Amin T, Ebmeier K P, Steele J D, Dougall N J, Best J, Migneco O, Nobili F, Scheidhauer K. Statistical parametric mapping of (99m)Tc-HMPAO-SPECT images for the diagnosis of Alzheimer's disease: normalizing to cerebellar tracer uptake.Neuroimage. 2002 November;17(3):1193-202.

Talairach J and Tournoux P, (1988). Co-Planar Stereotactic Atlas Of The human Brain. Stuttgart: Georg Thieme Verlag.

Worsley K J, Marrett S, Neelin P, Vandal A C, Friston K J, and Evans A C (1996). A unified statistical approach for determining significant voxels in images of cerebral activation. Human Brain Mapping, 4:58-73.

1. A method for producing an index indicative of brain disease comprising the steps of:
collecting positron emission tomographic image data showing metabolic activity in the brain of a patient;
spatially normalizing said image data using a standardized three dimensional coordinate system;
spatially filtering the normalized image data;
selecting specific regions of the brain showing extremes in metabolic activity;
collecting mean intensity values for the normalized, smoothed image data from said selected specific brain regions;
weighting said mean intensity values with standard weights derived from the group analysis used to create the standard; and
normalizing the ratio of said mean, weighted, metabolic activity image data to produce a numerical index.

2. The method according to claim 1 wherein the metabolic activity is indicated by glucose metabolism of brain cells.

3. The method according to claim 1 wherein the three-dimensional coordinate system is Talairach space.

4. The method according to claim 3 wherein the image data is transformed to conform in Talairach space using a twelve parameter, linear, affine algorithm.

5. The method according to claim 1 wherein the transformed image data is smoothed using an eight millimeter, isotropic, Gaussian filter kernel.

6. The method according to claim 1 wherein said normalized, smoothed image data is compared to data from age-matched patient controls using Standard Parametric Mapping techniques in a statistical group comparison.

7. The method according to claim 6 wherein the Standard Parametric Mapping is used to generate a map of the brain and the map is converted to a unit normal distribution Z score.

8. The method according to claim 7 wherein the Standard Parametric Mapping Z-score results are utilized to select specific regions of the brain showing extremes in metabolic activity.

9. The method according to claim 1 wherein statistical mapping procedures are utilized to create a plurality of three dimensional, identically sized, spherical volumes of interest.

10. The method according to claim 9 wherein mean intensity values for the volume elements are contained within each of said volumes of interest are determined wherein each said volume element is a cube of selected dimension.

11. The method according to claim 9 wherein each of a plurality of volumes of interest is placed at specific coordinates in said three dimensional coordinate system.

12. The method according to claim 9 wherein two sets of volumes of interest are selected, the first set being comprised of a plurality of volumes of interest with increased metabolism and the second set being comprised of a plurality of volumes of interest with decreased metabolism.

13. The method according to claim 12 wherein said first set of volumes of interest comprises four volumes of interest with increased metabolism and said second set of volumes of interest comprises nine volumes of interest with decreased metabolism.

14. The method according to claim 12 wherein the intensity values of said volumes of interest are used to create a first and second data set, said first data set comprising the ratios of the mean value of the intensities of the first set of volumes of interest with increased metabolism divided by the intensity values of each of the volumes of the second set of volumes of interest with decreased metabolism and said second data set comprising the ratios of each of the intensity values of the first set of volumes of interest with increased metabolism divided by the mean value of the intensities of the second set of volumes of interest with decreased metabolism.

15. The method according to claim 14 wherein the intensity values of the set of said thirteen volumes of interest are used to create a third and fourth data set, said third data set comprising the ratios of the mean of the intensity value of the set of four volumes of interest with increased metabolism divided by the intensity values of each of the nine volumes of interest with decreased metabolic activity and the fourth data set comprising the ratio of each of the intensity values of the set of four volumes of interest of increased metabolic activity divided by the mean value of the intensities of the volumes of interest with increased metabolic activity.

16. A method for diagnosing degenerative brain disease comprising the steps of:
collecting positron emission tomographic image data showing metabolic activity of a brain of a patient;
spatially normalizing said image data using a three dimensional coordinate system;
smoothing said normalized image data;
applying objective statistical analysis to select specific regions of the brain, said regions showing extreme changes in metabolic activity;
collecting mean intensity values for said normalized, smoothed image data from said selected specific regions;
weighting said mean intensity values based on a comparison of said mean intensity values taken from said patient to a set of mean intensity values of said specific brain region taken from a normal patient population; and
calculating an index using said weighted, mean intensity values wherein said index is a normalized ratio of said weighted, mean intensity values, taken from said sampled regions.

17. The method according to claim 1 wherein the disease detected is one or more of the following diseases:
Alzheimer's disease;
Parkinson's disease;
Huntington's disease;
Pick's Dementia;
Dementia with Lewy bodies;
Disease resulting from head injury;
Disease resulting from patient intake of drugs; and
Disease resulting from patient intake of alcohol.

18. The method according to claim 1 additionally comprising
using said weighted intensity values as a baseline reference for iterative optimization of each weighted intensity value;
forming a subset of weights taken from a control subject database said control subjects forming a first group;
maximally separating each of said weighted intensity values of each region taken from said patient from intensity values of analogous regions taken from control subjects using a dynamic table of patient weights and control subject weights wherein separations in intensity values between the patients and the normal controls are assessable in real time;
merging patient data with data from previous patients in a patient database to constitute a second group;
iteratively adjusting said weighted intensity values to maximize the separation between said patient and said control subjects while minimizing within-group variance; and
calculating a second Cognitive Decline Index utilizing the optimized weighted intensity values.
